(19) United States
(12) Patent Application Publication
(10) Pub. No.: US 2021/0197202 A1
(43) Pub. Date: Jul. 1, 2021

(54) MICROFLUIDIC SIPHONING ARRAY FOR NUCLEIC ACID QUANTIFICATION

(71) Applicant: Combinati Incorporated, Palo Alto, CA (US)

(72) Inventors: Andrew Zayac, Belmont, CA (US); Sammy Datwani, Pleasanton, CA (US); Megan Dueck, San Francisco, CA (US); Ju-Sung Hung, Palo Alto, CA (US)

(21) Appl. No.: 17/028,839

(22) Filed: Sep. 22, 2020

Related U.S. Application Data
(63) Continuation of application No. PCT/US2019/025539, filed on Apr. 3, 2019.
(60) Provisional application No. 62/652,859, filed on Apr. 4, 2018.

(51) Int. Cl. B01L 7/00; C12Q 1/686; B01L 3/00

(52) U.S. Cl. CPC B01L 7/52; C12Q 1/686; B01L 3/502715; B01L 2200/0689; B01L 2300/0609; B01L 2300/0663; B01L 2300/18

(57) ABSTRACT

The present disclose provides devices, methods and systems that may be used for amplifying and quantifying nucleic acid molecules. Methods for amplifying and quantifying nucleic acids may comprise isolating a sample comprising nucleic acid molecules into a plurality of chambers, performing a polymerase chain reaction on the plurality of chambers, and analyzing the results of the polymerase chain reaction.

### CROSS REFERENCE

This application is a continuation of U.S. Provisional Patent Application No. PCT/US2019/025539, filed Apr. 3, 2019, which claims the benefit of U.S. Provisional Application No. 62/652,859, filed on Apr. 4, 2018, which is entirely incorporated herein by reference.

### GOVERNMENT INTEREST STATEMENT

This invention was made with government support under Small Business Innovation Research grant number 1R430D023028-01 awarded by the National Institute of Health. The U.S. government has certain rights in the invention.

### BACKGROUND

Microfluidic devices are devices that contain structures that handle fluids on a small scale. Typically, a microfluidic device operates on a sub-millimeter scale and handles micro-liters, nano-liters, or smaller quantities of fluids. In microfluidic devices, a major fouling mechanism is trapped air, or bubbles, inside the micro-structure. This can be particularly problematic when using a thermoplastic material to create the microfluidic structure, as the gas permeability of thermoplastics is very low.

In order to avoid fouling by trapped air, previous microfluidic structures use either simple straight channel or branched channel designs with thermoplastic materials, or else manufacture the device using high gas permeability materials such as elastomers. However, simple designs limit possible functionality of the microfluidic device, and elastomeric materials are both difficult and expensive to manufacture, particularly at scale.

One application of microfluidic structures is in digital polymerase chain reaction (dPCR). dPCR dilutes a nucleic acid sample down to one or less nucleic acid template in each partition of a microfluidic structure providing an array of many partitions, and performs a PCR reaction across the array. By counting the partitions in which the template was successfully PCR amplified and applying Poisson statistics to the result, the target nucleic acid is quantified. Unlike the popular quantitative real-time PCR (qPCR) where templates are quantified by comparing the rate of PCR amplification of an unknown sample to the rate for a set of known qPCR standards, dPCR has proven to exhibit higher sensitivity, better precision and greater reproducibility.

For genomic researchers and clinicians, dPCR is particularly powerful in rare mutation detection, quantifying copy number variants, and Next Gen Sequencing library quantification. The potential use in clinical settings for liquid biopsy with cell free DNA and viral load quantification may further increase increases the value of dPCR technology. Existing dPCR solutions have used elastomeric valve arrays, silicon through-hole approaches, and microfluidic encapsulation of droplets in oil. Despite the growing number of available dPCR platforms, dPCR has been at a disadvantage when compared to the older qPCR technology which relies on counting the number of PCR amplification cycles. The combination of throughput, ease of use, performance and cost are the major barriers for gaining adoption in the market for dPCR.

### SUMMARY

Provided herein are methods, systems and devices that may be useful for amplifying and quantifying nucleic acids. The present disclosure provides methods, systems, and devices that may enable sample preparation, sample amplification, and sample analysis through the use of dPCR. This may enable a nucleic acid to be amplified and quantified at a reduced cost and complexity as compared to other systems and methods.

In aspect, the present disclosure provides a system for thermal cycling a microfluidic device, comprising: a holder that is configured to secure a microfluidic device comprising a plurality of chambers, wherein a chamber of the plurality of chambers is configured to contain a nucleic acid sample comprising at least one nucleic acid molecule; a thermal module that is configured to be in thermal communication with the chamber of the microfluidic device, wherein the thermal module is configured to thermal cycle the plurality of chambers such that a single round of thermal cycling is completed within about 20 seconds or less, and wherein the thermal module is configured to maintain a temperature across the plurality of chambers within about 0.2° C.; a pneumatic module in fluid communication with the microfluidic device when the microfluidic device is secured by the holder, wherein the pneumatic module is configured to (i) load the nucleic acid sample into the chamber, and (ii) apply pressure to the microfluidic device to maintain thermal contact between the microfluidic device and the thermal module; and one or more computer processors coupled to the thermal module and the pneumatic module, wherein the one or more computer processors are configured to (i) direct the pneumatic module to load the nucleic acid sample into the chamber, (ii) direct the pneumatic module to apply the pressure to the microfluidic device to maintain the thermal contact between the microfluidic device and the thermal module, and (iii) direct the thermal module to thermal cycle the plurality of chambers to amplify the at least one nucleic acid molecule in the chamber.

In some embodiments, the microfluidic device comprises a film or barrier that seals the chamber. In some embodiments, the film or barrier comprises a polymeric material. In some embodiments, the film or barrier has a thickness of less than or equal to about 250 micrometers (μm). In some embodiments, the film or barrier has a thickness of less than or equal to about 100 nm.

In some embodiments, the system further comprises an optical module in optical communication with the plurality of chambers, wherein the optical module is configured to image the plurality of chambers. In some embodiments, the film or barrier is configured to permit gas flow from the plurality of chambers to an environment external to the plurality of chambers under application of a pressure differential across the film or barrier. In some embodiments, the pneumatic module is configured to apply the pressure differential across the film or barrier. In some embodiments, the chamber has a volume of less than or equal to about 150 picoliters (pL). In some embodiments, the chamber has a volume of less than or equal to 100 pL. In some embodiments, the chamber has a cross-sectional dimension of less than or equal to about 100 nm. In some embodiments, the chamber has a depth of less than or equal to about 50 μm. In some embodiments, during use, a surface of the microfluidic device contacts the thermal module, and wherein the surface is substantially planar.

In some embodiments, the pneumatic module is configured to prevent warping of the microfluidic device during thermal cycling. In some embodiments, the thermal module is configured to maintain the temperature across the plurality of chambers within about 0.1° C. In some embodiments, the microfluidic device further comprises at least one channel in fluid communication with the plurality of chambers. In some embodiments, the microfluidic device further comprises a plurality of siphon apertures, and wherein the plurality of siphon apertures provide the fluid communication between the at least one channel and the plurality of chambers.

In another aspect, the present disclosure provides a system for thermal cycling a microfluidic device, comprising: a microfluidic device comprising a plurality of chambers, wherein a chamber of the plurality of chambers comprises a nucleic acid sample comprising at least one nucleic acid molecule, wherein the microfluidic device comprises a film or barrier sealing the chamber, and wherein the film or barrier has a thermal conductivity of less than or equal to about 1 watt per meter Kelvin (W/m-K) at 20° C.; a thermal module in thermal communication with the film or barrier of the microfluidic device, wherein the thermal module is configured to thermal cycle the microfluidic device such that a single round of thermal cycling is completed within about 20 seconds or less; and a pneumatic module in fluid communication with the microfluidic device, wherein the pneumatic module is configured to load the nucleic acid sample into the chamber, and wherein the pneumatic module is configured to apply pressure to the microfluidic device to maintain thermal contact between the film or barrier and the thermal module; and one or more computer processors coupled to the thermal module and the pneumatic module, wherein the one or more computer processors are configured to (i) direct the pneumatic module to load the nucleic acid sample into the chamber, (ii) direct the pneumatic module to apply the pressure to the microfluidic device to maintain the thermal contact between the film or barrier and the thermal module, and (iii) direct the thermal module to thermal cycle the plurality of chambers to amplify the at least one nucleic acid molecule in the chamber.

In some embodiments, the film or barrier comprises a polymeric material. In some embodiments, the film or barrier has a thermal conductivity of less than or equal to about 0.5 W/m-K at 20° C. In some embodiments, the film or barrier has a thermal conductivity of less than or equal to about 0.2 W/m-K at 20° C. In some embodiments, the system further comprises an optical module in optical communication with the plurality of chambers, wherein the optical module is configured to image the plurality of chambers. In some embodiments, the film or barrier has a thickness of less than or equal to about 250 micrometers (μm). In some embodiments, the film or barrier is configured to permit gas flow from the plurality of chambers to an environment external to the plurality of chambers under application of a pressure differential across the film or barrier.

In some embodiments, the pneumatic module is configured to apply the pressure differential across the film or barrier. In some embodiments, the chamber has a volume of less than or equal to about 150 picoliters (pL). In some embodiments, the chamber has a volume of less than or equal to 100 pL. In some embodiments, the chamber has a cross-sectional dimension of less than or equal to about 100 μm. In some embodiments, the chamber has a depth of less than or equal to about 50 μm. In some embodiments, the film or barrier contacts a surface of the thermal module. In some embodiments, the film or barrier is substantially planar. In some embodiments, the pneumatic module is configured to prevent warping of the microfluidic device during thermal cycling. In some embodiments, the microfluidic device further comprises at least one channel in fluid communication with the plurality of chambers. In some embodiments, the microfluidic device further comprises a plurality of siphon apertures, and wherein the plurality of siphon apertures provide the fluid communication between the at least one channel and the plurality of chambers.

In another aspect, the present disclosure provides a method for thermal cycling a microfluidic device, comprising: providing a holder that secures a microfluidic device comprising a plurality of chambers, a thermal module that is in thermal communication with the microfluidic device, and a pneumatic module that is in fluid communication with the microfluidic device; using the pneumatic module to load a nucleic acid sample comprising at least one nucleic acid molecule into a chamber of the plurality of chambers of the microfluidic device; using the pneumatic module to apply pressure to the microfluidic device to maintain thermal contact between the microfluidic device and the thermal module; and using the thermal module to thermal cycle the plurality of chambers to amplify the at least one nucleic acid molecule in the chamber, wherein a single round of thermal cycling is completed within about 20 seconds or less, and wherein the thermal module maintains a temperature across the plurality of chambers within about 0.2° C.

In some embodiments, thermal cycling the plurality of chambers activates a polymerase chain reaction. In some embodiments, at least forty cycles of the polymerase chain reaction are completed in less than twenty minutes. In some embodiments, the microfluidic device comprises a film or barrier that seals the chamber. In some embodiments, the film or barrier is a polymeric material. In some embodiments, the film or barrier has a thickness of less than or equal to about 250 micrometers (μm). In some embodiments, the film or barrier permits gas flow from the plurality of chambers to an environment external to the plurality of chambers under application of a pressure differential across the film or barrier. In some embodiments, the method further comprises using the pneumatic module to apply the pressure differential across the film or barrier.

In some embodiments, the film or barrier contacts a surface of the thermal module. In some embodiments, the method further comprises using an optical module in optical communication with the plurality of chambers to image the plurality of chambers. In some embodiments, the chamber has a depth of less than or equal to about 50 μm. In some embodiments, using the pneumatic module to apply pressure to the microfluidic device prevents warping of the microfluidic device during thermal cycling. In some embodiments, the thermal module maintains the temperature across the plurality of chambers within about 0.1° C.

In some embodiments, the microfluidic device further comprises at least one channel in fluid communication with the plurality of chambers. In some embodiments, the microfluidic device further comprises a plurality of siphon apertures, and wherein the plurality of siphon apertures provide the fluid communication between the at least one channel and the plurality of chambers. In some embodiments, the method further comprises using the pneumatic module to apply a first pressure to the at least one channel to load a sample into the at least one channel. In some embodiments, the method further comprises using the pneumatic module to apply a second pressure to the at least one channel to load the sample into the plurality of chambers. In some embodiments, the microfluidic device comprises a film or barrier that seals the plurality of chambers, and wherein the second pressure is sufficient to permit gas flow from the plurality of chambers through the film or barrier to an environment external to the plurality of chambers.

In another aspect, the present disclosure provides a method for thermal cycling a microfluidic device, comprising: providing a microfluidic device that is in fluid communication with a pneumatic module and that is in thermal communication with a thermal module, wherein the microfluidic device comprises a plurality of chambers, wherein the microfluidic device comprises a film or barrier that seals the plurality of chambers, wherein the film or barrier has a thermal conductivity of less than or equal to about 1 watt per meter Kelvin (W/m-K) at 20° C., and wherein the film or barrier is in thermal communication with the thermal module; using the pneumatic module to load a nucleic acid sample comprising at least one nucleic acid molecule into a chamber of the plurality of chambers of the microfluidic device; using the pneumatic module to apply a pressure to the microfluidic device to maintain thermal contact between the film or barrier of the microfluidic device and the thermal module; and using the thermal module to thermal cycle the plurality of chambers to amplify the at least one nucleic acid molecule in the chamber, wherein a single round of thermal cycling is completed within about 20 seconds or less.

In some embodiments, thermal cycling the plurality of chambers activates a polymerase chain reaction. In some embodiments, at least forty cycles of the polymerase chain reaction are completed in less than twenty minutes. In some embodiments, the film or barrier has a thermal conductivity of less than or equal to about 0.5 W/m-K at 20° C. In some embodiments, the film or barrier has a thermal conductivity of less than or equal to about 0.2 W/m-K at 20° C. In some embodiments, the film or barrier comprises a polymeric material. In some embodiments, the method further comprises using an optical module in optical communication with the plurality of chambers to image the plurality of chambers. In some embodiments, the film or barrier has a thickness of less than or equal to about 250 micrometers (μm).

In some embodiments, the film or barrier permits gas flow from the plurality of chambers to an environment external to the plurality of chambers under application of a pressure differential across the film or barrier. In some embodiments, the method further comprises using the pneumatic module to apply the pressure differential across the film or barrier. In some embodiments, a chamber of the plurality of chambers has a depth of less than or equal to about 50 μm. In some embodiments, the film or barrier contacts a surface of the thermal module. In some embodiments, using the pneumatic module to apply pressure to the microfluidic device prevents warping of the microfluidic device during thermal cycling. In some embodiments, the microfluidic device further comprises at least one channel in fluid communication with the plurality of chambers. In some embodiments, the microfluidic device further comprises a plurality of siphon apertures, and wherein the plurality of siphon apertures provide the fluid communication between the at least one channel and the plurality of chambers. In some embodiments, the method further comprises using the pneumatic module to apply a first pressure to the at least one channel to load a sample into the at least one channel. In some embodiments, the method further comprises using the pneumatic module to apply a second pressure to the at least one channel to load the sample into the plurality of chambers. In some embodiments, the second pressure is sufficient to permit gas flow from the plurality of chambers through the film or barrier to an environment external to the plurality of chambers.

In another aspect, the present disclosure provides a microfluidic device comprising a plurality of chambers, wherein a chamber of the plurality of chambers is configured to contain a nucleic acid sample comprising at least one nucleic acid molecule, wherein the plurality of chambers is configured to (i) undergo thermal cycling at a rate of about 20 seconds or less and (ii) provide a temperature uniformity at a deviation within about 0.2° C., wherein the microfluidic device comprises a film or barrier configured to seal the chamber during thermal cycling.

In some embodiments, the film or barrier is configured to permit gas flow from the plurality of chambers to an environment external to the plurality of chambers under application of a pressure differential across the film or barrier. In some embodiments, the microfluidic device further comprises at least one channel in fluid communication with the plurality of chambers. In some embodiments, the microfluidic device further comprises a plurality of siphon apertures, and wherein the plurality of siphon apertures provide the fluid communication between the at least one channel and the plurality of chambers.

In another aspect, the present disclosure provides a microfluidic device comprising a plurality of chambers, wherein a chamber of the plurality of chambers is configured to contain a nucleic acid sample comprising at least one nucleic acid molecule, wherein the plurality of chambers is configured to undergo thermal cycling at a rate of about 20 seconds or less, wherein the microfluidic device comprises a film or barrier configured to seal the chamber during thermal cycling, and wherein the film or barrier has a thermal conductivity of less than or equal to about 1 watt per meter Kelvin (W/m-K) at 20° C.

In some embodiments, the film or barrier is configured to permit gas flow from the plurality of chambers to an environment external to the plurality of chambers under application of a pressure differential across the film or barrier. In some embodiments, the microfluidic device further comprises at least one channel in fluid communication with the plurality of chambers. In some embodiments, the microfluidic device further comprises a plurality of siphon apertures, wherein the plurality of siphon apertures provide the fluid communication between the at least one channel and the plurality of chambers.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIGS. 1A and 1B illustrate an example of a microfluidic structure; FIG. 1A shows the structure from an overhead view, while FIG. 1B illustrates a cross-section of the structure;

FIGS. 2A and 2B schematically illustrates example arrangements of chambers, siphon apertures, and channels within a microfluidic device; FIG. 2A shows an embodiment in which parallel sub-channels and one or more cross-channels are used to form a grid of chambers; FIG. 2B shows an embodiment in which a single channel in a serpentine pattern forms a hexagonal grid of chambers;

FIGS. 3A-3D show methods for use of an example microfluidic device; FIG. 3A shows a step of applying reagent at low pressure; FIG. 3B shows a step of applying a pressure differential across the microfluidic device to force partitioning and outgassing; FIG. 3C shows a step of applying fluid at low pressure to clear the channel; FIG. 3D shows the state of the system after the completion of the method;

FIG. 4 schematically illustrates a method of manufacture of a microfluidic device;

FIG. 5 schematically illustrates an example digital PCR process to be employed with the a microfluidic device;

FIG. 6 schematically illustrates a machine for performing the a nucleic acid amplification and quantification method in a single machine;

FIG. 7 schematically illustrates an example computer control system that is programmed or otherwise configured to implement methods provided herein;

FIGS. 8A and 8B show the microfluidic device and sample partitioning; FIG. 8A shows a microfluidic device formed by micromolding a thermoplastic; FIG. 8B show fluorescent images of the sample partitioning process;

FIG. 9 shows an example system for processing a nucleic acid sample;

FIGS. 10A-10D show two color (one color representing sample signal and the other representing a normalization signal) fluorescent detection of nucleic acid amplification of partitions containing approximately one nucleic acid template copy on average and partitions containing zero nucleic acid template copies (no template control or NTC); FIG. 10A shows zero copies per partition (NTC) after amplification; FIG. 10B shows nucleic acid amplification of partitions containing approximately one copy per partition; FIG. 10C shows a plot of NTC fluorescence intensity of both fluorescent colors; and FIG. 10D shows a plot of fluorescence intensity of both fluorescent colors of the amplified sample;

FIG. 11A illustrates an example microfluidic device that may be used for rapid thermal cycling; FIG. 11B shows a cross sectional view of an example microfluidic device that may be used for rapid thermal cycling;

FIGS. 12A and 12B show an example system for rapid thermal cycling; FIG. 12A shows an example system for rapid thermal cycling with a pneumatic manifold not contacting the microfluidic device; FIG. 12B shows an example system for rapid thermal cycling with a pneumatic manifold contacting the microfluidic device; and

FIGS. 13A-13E show example polymerase chain reaction results; FIG. 13A shows a plot of template concentration as a function of thermal cycling dwell time; FIG. 13B shows fluorescent detection of template concentration after a polymerase chain reaction cycle; FIG. 13C shows fluorescent detection of template concentration after reducing dwell times by fifty percent; FIG. 13D shows fluorescent detection of template concentration after reducing dwell times by seventy-five percent; FIG. 13E shows fluorescent detection of template concentration after reducing dwell times by eighty-five percent.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

As used herein, the terms “amplification” and “amplify” are used interchangeably and generally refer to generating one or more copies or “amplified product” of a nucleic acid. Such amplification may be using polymerase chain reaction (PCR) or isothermal amplification, for example.

As used herein, the term “nucleic acid” generally refers to a polymeric form of nucleotides of any length (e.g., at least 2, 3, 4, 5, 6, 7, 8, 9, 10, 100, 500, or 1000 nucleotides), either deoxyribonucleotides or ribonucleotides, or analogs thereof. A nucleic acid may include one or more subunits selected from adenosine (A), cytosine (C), guanine (G), thymine (TO, and uracil (U), or variants thereof. A nucleotide can include A, C, G, T, or U, or variants thereof. A nucleotide can include any subunit that can be incorporated into a growing nucleic acid strand. Such subunit can be A, C, G, T, or U, or any other subunit that is specific to one of more complementary A, C, G, T, or U, or complementary to a purine (i.e., A or G, or variant thereof) or pyrimidine (i.e., C, T, or U, or variant thereof). In some examples, a nucleic acid may be single-stranded or double stranded, in some cases, a nucleic acid molecule is circular. Non-limiting examples of nucleic acids include DNA and RNA. Nucleic acids can include coding or non-coding regions of a gene or gene fragment, loci (locus) defined from linkage analysis, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant nucleic acids, branched nucleic acids, plasmids, vectors, isolated DNA of any sequence, isolated RNA of any sequence, nucleic acid probes, and primers. A nucleic acid may comprise one or more modified nucleotides, such as methylated nucleotides and nucleotide analogs.

As used herein, the terms “polymerase chain reaction reagent” or “PCR reagent” may be used interchangeably and generally refer to a composition comprising reagents necessary to complete a nucleic acid amplification reaction (e.g., DNA amplification), with non-limiting examples of such reagents including primer sets or priming sites (e.g., nick) having specificity for a target nucleic acid, polymerases, suitable buffers, co-factors (e.g., divalent and monovalent cations), dNTPs, and other enzymes. A PCR reagent may also include probes, indicators, and molecules that comprise probes and indicators.

As used herein, the term “probe” generally refers to a molecule that comprises a detectable moiety, the presence or absence of which may be used to detect the presence or absence of an amplified product. Non-limiting examples of detectable moieties may include radiolabels, stable isotope labels, fluorescent labels, chemiluminescent labels, enzymatic labels, colorimetric labels, or any combination thereof.

As used herein, the term “extension” generally refers to incorporation of nucleotides into a nucleic acid in a template directed fashion. Extension may occur via the aid of an enzyme. For example, extension may occur via the aid of a polymerase. Conditions at which extension may occur include an “extension temperature” that generally refers to a temperature at which extension is achieved and an “extension duration” that generally refers to an amount of time allotted for extension to occur.

As used herein, the term “indicator molecule” generally refers to a molecule that comprises a detectable moiety, the presence or absence of which may be used to indicate sample partitioning. Non-limiting examples of detectable moieties may include radiolabels, stable isotope labels, fluorescent labels, chemiluminescent labels, enzymatic labels, colorimetric labels, or any combination thereof.

The term “sample,” as used herein, generally refers to any sample containing or suspected of containing a nucleic acid molecule. For example, a sample can be a biological sample containing one or more nucleic acid molecules. The biological sample can be obtained (e.g., extracted or isolated) from or include blood (e.g., whole blood), plasma, serum, urine, saliva, mucosal excretions, sputum, stool and tears. The biological sample can be a fluid or tissue sample (e.g., skin sample). In some examples, the sample is obtained from a cell-free bodily fluid, such as whole blood. In such instance, the sample can include cell-free DNA and/or cell-free RNA. In some examples, the sample can include circulating tumor cells. In some examples, the sample is an environmental sample (e.g., soil, waste, ambient air and etc.), industrial sample (e.g., samples from any industrial processes), and food samples (e.g., dairy products, vegetable products, and meat products). The sample may be processed prior to loading into the microfluidic device. For example, the sample may be processed to lyse cells, purify the nucleic acid molecules, and/or to include reagents. Alternatively, or in addition to, the sample may not be processed prior to loading into the microfluidic device.

As used herein, the term “fluid” generally refers to a liquid or a gas. A fluid cannot maintain a defined shape and will flow during an observable time frame to fill the container into which it is put. Thus, the fluid may have any suitable viscosity that permits flow. If two or more fluids are present, each fluid may be independently selected among essentially any fluids (liquids, gases, and the like) by those of ordinary skill in the art.

As used herein, the term “partition” generally refers to a division into or distribution into portions or shares. For example, a partitioned sample is a sample that is isolated from other samples. Examples of structures that enable sample partitioning include wells and chambers.

As used herein, the term “microfluidic” generally refers to a chip, area, device, article, or system including at least one channel (e.g., or at least one microchannel), a plurality of siphon apertures, and an array of chambers (e.g., or an array of microchambers). The channel may have a cross-sectional dimension less than or equal to about 10 millimeters (mm), less than or equal to about 5 mm, less than or equal to about 4 mm, less than or equal to about 3 mm, less than or equal to about 2 mm, less than or equal to about 1.5 mm, less than or equal to about 1 mm, less than or equal to about 750 micrometers (μm), less than or equal to about 500 μm, less than or equal to about 250 μm, less than or equal to about 100 μm, or less.

As used herein, the term “depth” generally refers to the distance measured from the bottom of the channel or microchannel, siphon aperture, or chamber or microchamber to the thin film that caps the channel, plurality of siphon apertures, and array of chambers.

As used herein, the terms “cross-section” or “cross-sectional” may be used interchangeably and generally refer to a dimension or area of a channel or microchannel or siphon aperture that is substantially perpendicularly to the long dimension of the feature.

As used herein, the terms “pressurized off-gassing” or “pressurized degassing” may be used interchangeably and generally refer to removal or evacuation of a gas (e.g., air, nitrogen, oxygen, etc.) from a channel or chamber of the device (e.g., microfluidic device) to an environment external to the channel or chamber through the application of a pressure differential. The pressure differential may be applied between the channel or chamber and the environment external to the channel or chamber. The pressure differential may be provided by the application of a pressure source to one or more inlets to the device or application of a vacuum source to one or more surfaces of the device. Pressurized off-gassing or pressurized degassing may be permitted through a film or membrane covering one or more sides of the channel or chamber.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The present disclosure describes a microfluidic device formed out of a polymer (e.g., thermoplastic) and incorporating a thin film, membrane, or other barrier to allow for pressurized outgassing or degassing while serving as a gas barrier when pressure is released. The use of polymers (e.g., thermoplastics) to form the microfluidic structure may allow for the use of an inexpensive and highly scalable injection molding processes, while the thin film may provide the ability to outgas via pressurization, avoiding the fouling problems that may be present some microfluidic structures that do not incorporate such thin films.

One use for this structure is a microfluidic design incorporating an array of dead-ended chambers (e.g., microchambers) connected by channels (e.g., microchannels), formed out of thermoplastics. This design can be used in a dPCR application to partition reagents into the array of chambers and thereby used to quantify nucleic acids in dPCR.

### Microfluidic Device for Analyzing Nucleic Acid Samples

In an aspect, the present disclosure provides a microfluidic device comprising a plurality of chambers. A chamber to the plurality of chambers may be configured to contain or may contain a nucleic acid sample comprising at least one nucleic acid molecule. The plurality of chambers may be configured to undergo thermal cycling at a rate of about 20 seconds or less. The microfluidic device may comprise a film or barrier. The film or barrier may be configured to seal or may seal the chamber(s) during thermal cycling. The film or barrier may have a thermal conductivity of less than or equal to about 1 watt per meter Kelvin (W/m-K).

In another aspect, the present disclosure provides a microfluidic device comprising a plurality of chambers. A chamber of the plurality of chambers may be configure to contain or may contain a nucleic acid sample comprising at least one nucleic acid molecule. The plurality of chambers may be configured to undergo thermal cycling at a rate of about 20 seconds or less and provide a temperature uniformity at a deviation within about 0.2° C. the microfluidic device may comprise a film or barrier that seals the chamber(s) during thermal cycling.

In another aspect, the present disclosure provides a microfluidic device for analyzing nucleic acid samples. The device may comprise a channel (e.g., microchannel) connected to an inlet and an outlet. The microfluidic device may also include a plurality of chambers (e.g., microchambers) and a plurality of siphon apertures. The plurality of chambers may be connected to the channel by the plurality of siphon apertures. The microfluidic device may include a film (e.g., thermoplastic thin film), barrier, or membrane which caps and/or covers and seals (e.g., hermetically seals) the channel, chambers, and/or siphon apertures. The film, barrier, or membrane may be at least partially gas permeable when a pressure differential is applied across the film, barrier, or membrane.

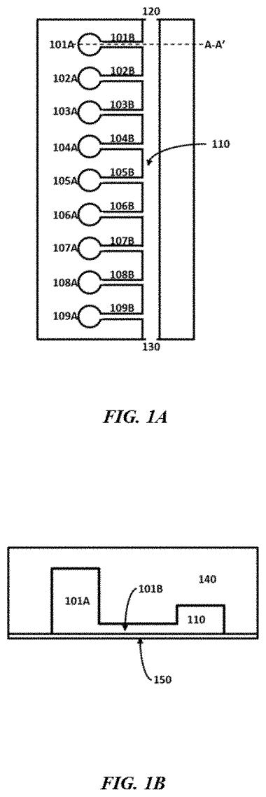

FIGS. 1A and 1B show examples of a microfluidic structure according to certain embodiments of the present disclosure. FIG. 1A shows an example microfluidic device from a top view. The microfluidic device comprises a channel 110, with an inlet 120, and an outlet 130. The channel is connected to a plurality of siphon apertures 101B—109B. The plurality of siphon apertures connects the channel to a plurality of chambers 101A-109A. FIG. 1B shows a cross-sectional view of a single chamber along the dashed line marked A-A′. The single chamber 101A is connected to the channel 110 by a siphon aperture 101B. The microfluidic device body 140 may be formed from a rigid plastic material. The microstructures of the microfluidic device may be capped and sealed by a thin film 150. The thin film may be gas impermeable when a small pressure differential is applied across the film and gas permeable when a large pressure differential is applied across the film. This may allow for outgassing through the thin film when a pressure is applied to the interior structure of the microfluidic device. In an alternative embodiment, outgassing may occur when a vacuum is applied external to the microfluidic device.

The film, barrier, or membrane may comprise a polymer or polymeric material, such as a thermoplastic. The polymer may be configured such that it permits gasses to flow across the film, barrier, or membrane when a pressure differential is applied to the film, barrier, or membrane. The film, barrier, or membrane may not permit water or water vapor to flow across the film, barrier, or membrane when the pressure differential is applied. When a pressure differential is not applied, the film, barrier, or membrane may not permit gas flow (e.g., outgassing) of the chambers, siphon apertures, and/or channel. The film, barrier, or membrane may be configured to permit, or may permit, transfer of thermal energy across the film, barrier, or membrane. The film, barrier, or membrane may comprise a material with a low thermal conductivity (e.g., non-thermally conductive polymer). Alternatively, or in addition to, the film, barrier, or membrane may comprise a material with a high thermal conductivity (e.g., metal film, etc.). The film, barrier, or membrane may seal a single chamber. Alternatively, or in addition to, the film, barrier, or membrane may seal multiple chambers. The film, barrier, or membrane may be disposed on a surface of the microfluidic device to seal the chamber or chambers. The film, barrier, or membrane may be substantially planer or planer. The surface of the microfluidic device may be substantially planer or planer. The substantially planer or planer surface or film may contact a thermal module during thermal cycling and transfer of thermal energy from the thermal module (e.g., heater, heating element, Peltier etc.) to the chambers.

Transfer of thermal energy may be permitted due to the thickness of the film, barrier, or membrane (e.g., less than 250 micrometers (μm) thick). The film, barrier, or membrane may have a thermal conductivity that is less than or equal to about 5 Watts per meters Kelvin (W/m-K), 4 W/m-K, 3 W/m-K, 2 W/m-K, 1.5 W/m-K, 1 W/m-K, 0.8 W/m-K, 0.6 W/m-K, 0.4 W/m-K, 0.2 W/m-K, 0.1 W/m-K, or less at 20° C. The film, barrier, or membrane may have a thermal conductivity that is from about 0.1 W/m-K to 0.2 W/m-K, 0.1 W/m-K to 0.4 W/m-K, 0.1 W/m-K to 0.6 W/m-K, 0.1 W/m-K to 0.8 W/m-K, 0.1 W/m-K to 1 W/m-K, 0.1 W/m-K to 1.5 W/m-K, 0.1 W/m-K to 2 W/m-K, 0.1 W/m-K to 3 W/m-K, 0.1 W/m-K to 4 W/m-K, 0.1 W/m-K to 5 W/m-K, 0.2 W/m-K to 0.4 W/m-K, 0.2 W/m-K to 0.6 W/m-K, 0.2 W/m-K to 0.8 W/m-K, 0.2 W/m-K to 1 W/m-K, 0.2 W/m-K to 1.5 W/m-K, 0.2 W/m-K to 2 W/m-K, 0.2 W/m-K to 3 W/m-K, 0.2 W/m-K to 4 W/m-K, 0.2 W/m-K to 5 W/m-K, 0.4 W/m-K to 0.6 W/m-K, 0.4 W/m-K to 0.8 W/m-K, 0.4 W/m-K to 1 W/m-K, 0.4 W/m-K to 1.5 W/m-K, 0.4 W/m-K to 2 W/m-K, 0.4 W/m-K to 3 W/m-K, 0.4 W/m-K to 4 W/m-K, 0.4 W/m-K to 5 W/m-K, 0.6 W/m-K to 0.8 W/m-K, 0.6 W/m-K to 1 W/m-K, 0.6 W/m-K to 1.5 W/m-K, 0.6 W/m-K to 2 W/m-K, 0.6 W/m-K to 3 W/m-K, 0.6 W/m-K to 4 W/m-K, 0.6 W/m-K to 5 W/m-K, 0.8 W/m-K to 1 W/m-K, 0.8 W/m-K to 1.5 W/m-K, 0.8 W/m-K to 2 W/m-K, 0.8 W/m-K to 3 W/m-K, 0.8 W/m-K to 4 W/m-K, 0.8 W/m-K to 5 W/m-K, 1 W/m-K to 1.5 W/m-K, 1 W/m-K to 2 W/m-K, 1 W/m-K to 3 W/m-K, 1 W/m-K to 4 W/m-K, 1 W/m-K to 5 W/m-K, 1.5 W/m-K to 2 W/m-K, 1.5 W/m-K to 3 W/m-K, 1.5 W/m-K to 4 W/m-K, 1.5 W/m-K to 5 W/m-K, 2 W/m-K to 3 W/m-K, 2 W/m-K to 4 W/m-K, 2 W/m-K to 5 W/m-K, 3 W/m-K to 4 W/m-K, 3 W/m-K to 5 W/m-K, or 4 W/m-K to 5 W/m-K at 20° C. In an example, the thermal conductivity of the film, barrier, or membrane is less than or equal to about 1 W/m-K at 20° C. In another example, the thermal conductivity of the film, barrier, or membrane is less than or equal to about 0.5 W/m-K at 20° C. In another example, the thermal conductivity of the film, barrier, or membrane is less than or equal to about 0.2 W/m-K at 20° C. In another example, the thermal conductivity of the film, barrier, or membrane is from about 0.1 W/m-K to 0.5 W/m-K at 20° C. In an example, the film, barrier, or membrane has a thermal conductivity of less than about 0.15 W/m-K at 20° C.

The gas permeability of the film, barrier, or membrane may be induced by elevated pressures. In some embodiments the pressure induced gas permeable thin film may cover the array of chambers and the channel and siphon apertures may be covered by a non-gas permeable film. In some embodiments, the pressure induced gas permeable thin film may cover the array of chambers and the siphon apertures and the channel may be covered by a non-gas permeable film. Alternatively, the pressure induced gas permeable thin film may cover the array of chambers, the siphon apertures, and the channel. In some embodiments, the thickness of the thin film may be less than or equal to about 500 micrometers (μm), less than or equal to about 250 μm, less than or equal to about 200 μm, less than or equal to about 150 μm, less than or equal to about 100 μm, less than or equal to about 75 μm, less than or equal to about 50 μm, less than or equal to about 25 μm, or less. In some embodiments, the thickness of the thin film may be from about 0.1 μm to about 200 μm or about 0.5 μm to about 150 μm. In some examples, the thickness of the thin film may be from about 50 μm to about 200 μm. In some examples, the thickness of the thin film may be from about 100 μm to about 200 μm. In some examples, the thickness of the thin film is about 100 μm to about 150 μm. In an example, the thin film is approximately 100 μm in thickness. The thickness of the film may be selected by manufacturability of the thin film, the air permeability of the thin film, the volume of each partition to be out-gassed, the available pressure, and/or the desired time to complete the siphoning process.

In some embodiments, the microfluidic device may comprise a single array of chambers. In some embodiments, the microfluidic device may comprise multiple arrays of chambers, each array of chambers isolated from the others. The arrays of chambers may be arranged in a row, in a grid configuration, in an alternating pattern, or in any other configuration. In some embodiments, the microfluidic device may have at least about 1, at least about 2, at least about 3, at least about 4, at least about 5, at least about 10, at least about 15, at least about 20, at least about 30, at least about 40, at least about 50, or more arrays of chambers. In some embodiments, the arrays of chambers are identical. In some embodiments, the microfluidic device may comprise multiple arrays of chambers that are not identical. The arrays of chambers may all have the same external dimension (i.e., the length and width of the array of chambers that encompasses all features of the array of chambers) or the arrays of chambers may have different external dimensions.

In some embodiments, an array of chambers may have a width of at most about 100 mm, about 75 mm, about 50 mm, about 40 mm, about 30 mm, about 20 mm, about 10 mm, about 8 mm, about 6 mm, about 4 mm, about 2 mm, about 1 mm, or less. The array of chambers may have a length of at most about 50 mm, about 40 mm, about 30 mm, about 20 mm, about 10 mm, about 8 mm, about 6 mm, about 4 mm, about 2 mm, 1 mm, or less. The width may be from about 1 mm to 100 mm, or 10 mm to 50 mm. The length may be from about 1 mm to 50 mm, or 5 mm to 20 mm.

In some examples, the array of chambers may have a width of about 100 mm and a length of about 40 mm. In some examples, the array of chambers may have a width of about 80 mm and a length of about 30 mm. In some examples, the array of chambers may have a width of about 60 mm and a length of about 25 mm. In some examples, the array of chambers may have a width of about 40 mm and a length of about 15 mm. In some examples, the array of chambers may have a width of about 30 mm and a length of about 10 mm. In some examples, the array of chambers may have a width of about 20 mm and a length of about 8 mm. In some examples, the array of chambers may have a width of about 10 mm and a length of about 4 mm. The external dimension may be determined by the total number of chambers desired, the dimension of each chamber, and the minimum distance between each chamber for manufacturability.

In some embodiments, the channel is substantially parallel to the long dimension of the microfluidic device. In some embodiments, the channel may be substantially perpendicular to the long dimension of the microfluidic device. In some embodiments, the channel may be neither substantially parallel nor substantially perpendicular to the long dimension of the microfluidic device. The angle between the channel and the long dimension of the microfluidic device may be at least about 5°, at least about 10°, at least about 15°, at least about 20°, at least about 30°, at least about 40°, at least about 50°, at least about 60°, at least about 70°, at least about 90°, at least about 100°, at least about 110°, at least about 120°, at least about 130°, at least about 140°, at least about 150°, at least about 160°, or at least about 170°. In some embodiments, the channel may be a single long channel. In some embodiments, the channel may have bends, curves, or angles. The channel may have a long dimension that is less than or equal to 100 mm, less than or equal to about 75 mm, less than or equal to about 50 mm, less than or equal to about 40 mm, less than or equal to about 30 mm, less than or equal to about 20 mm, less than or equal to about 10 mm, less than or equal to about 8 mm, less than or equal to about 6 mm, less than or equal to about 4 mm, less than or equal to about 2 mm, or less. The length of the channel may be bounded by the external length or width of the microfluidic device. The channel may have a depth of less than or equal to about 500 μm, less than or equal to about 250 μm, less than or equal to about 100 μm, less than or equal to about 80 μm, less than or equal to about 60 μm, less than or equal to about 30 μm, less than or equal to about 20 μm, less than or equal to about 10 μm, or less. The channel may have a cross-sectional dimension (e.g., width) of less than or equal to about 500 μm, less than or equal to about 250 μm, less than or equal to about 100 μm, less than or equal to about 75 μm, less than or equal to about 50 μm, less than or equal to about 40 μm, less than or equal to about 30 μm, less than or equal to about 20 μm, less than or equal to about 10 μm, or less.

In some examples, the cross-sectional dimensions of the channel may be about 100 μm wide by about 100 μm deep. In some examples, the cross-sectional dimensions of the channel may be about 100 μm wide by about 80 μm deep. In some examples, the cross-sectional dimensions of the channel may be about 100 μm wide by about 60 μm deep. In some examples, the cross-sectional dimensions of the channel may be about 100 μm wide by about 40 μm deep. In some examples, the cross-sectional dimensions of the channel may be about 100 μm wide by about 20 μm deep. In some examples, the cross-sectional dimensions of the channel may be about 100 μm wide by about 10 μm deep. In some examples, the cross-sectional dimensions of the channel may be about 80 μm wide by about 100 μm deep. In some examples, the cross-sectional dimensions of the channel may be about 60 μm wide by about 100 μm deep. In some examples, the cross-sectional dimensions of the channel may be about 40 μm wide by about 100 μm deep. In some examples, the cross-sectional dimensions of the channel may be about 20 μm wide by about 100 μm deep. In some examples, the cross-sectional dimensions of the channel may be about 10 μm wide by about 100 μm deep. In some examples, the cross-sectional dimensions of the channel may be about 80 μm wide by about 80 μm deep. In some examples, the cross-sectional dimensions of the channel may be about 60 μm wide by about 60 μm deep. In some examples, the cross-sectional dimensions of the channel may be about 40 μm wide by about 40 μm deep. In some examples, the cross-sectional dimensions of the channel may be about 20 μm wide by about 20 μm deep. In some examples, the cross-sectional dimensions of the channel may be about 10 μm wide by about 10 μm deep. The cross-sectional shape of the channel may be any suitable cross-sectional shape including, but not limited to, circular, oval, triangular, square, or rectangular. The cross-sectional area of the channel may be constant along the length of the channel. Alternatively, or in addition to, the cross-sectional area of the channel may vary along the length of the channel. The cross-sectional area of the channel may vary between about 50% and 150%, between about 60% and 125%, between about 70% and 120%, between about 80% and 115%, between about 90% and 110%, between about 95% and 100%, or between about 98% and 102%. The cross-sectional area of the channel may be less than or equal to about 10,000 micrometers squared (μm²), less than or equal to about 7,500 μm², less than or equal to about 5,000 μm², less than or equal to about 2,500 μm², less than or equal to about 1,000 μm², less than or equal to about 750 μm², less than or equal to about 500 μm², less than or equal to about 400 μm², less than or equal to about 300 μm², less than or equal to about 200 μm², less than or equal to about 100 μm², or less.

In some embodiments, the channel may have a single inlet and a single outlet. Alternatively, the channel may have multiple inlets, multiple outlets, or multiple inlets and multiple outlets. The inlets and outlets may have the same diameter or they may have different diameters. The inlets and outlets may have diameters less than or equal to about 2.5 millimeters (mm), less than or equal to about 2 mm, less than or equal to about 1.5 mm, less than or equal to about 1 mm, less than about 0.5 mm, or less.

In some embodiments, the array of chambers may have at least about 1,000 chambers, at least about 5,000 chambers, at least about 10,000 chambers, at least about 20,000 chambers, at least about 30,000 chambers, at least about 40,000 chambers, at least about 50,000 chambers, at least about 100,000 chambers, or more. In some examples, the microfluidic device may have from about 10,000 to about 30,000 chambers. In some examples, the microfluidic device may have from about 15,000 to about 25,000 chambers. The chambers may be cylindrical in shape, hemispherical in shape, or a combination of cylindrical and hemispherical in shape. The chambers may have diameters of less than or equal to about 500 μm, less than or equal to about 250 μm, less than or equal to about 100 μm, less than or equal to about 80 μm, less than or equal to about 60 μm, less than or equal to about 30 μm, less than or equal to about 15 μm, or less. The depth of the chambers may be less than or equal to about 500 μm, less than or equal to about 250 μm, less than or equal to about 100 μm, less than or equal to about 80 μm, less than or equal to about 60 μm, less than or equal to about 30 μm, less than or equal to about 15 μm, or less. In some examples, the chambers may have a diameter of about 30 μm and a depth of about 100 μm. In some examples, the chambers may have a diameter of about 35 μm and a depth of about 80 μm. In some examples, the chambers may have a diameter of about 40 μm and a depth of about 70 μm. In some examples, the chambers may have a diameter of about 50 μm and a depth of about 60 μm. In some examples, the chambers may have a diameter of about 60 μm and a depth of about 40 μm. In some examples, the chambers may have a diameter of about 80 μm and a depth of about 35 μm. In some examples, the chambers may have a diameter of about 100 μm and a depth of about 30 μm. In some embodiments, the chambers and the channel have the same depth. In an alternative embodiment, the chambers and the channel have different depths.

The chambers (e.g., microchambers) may have any volume. The chambers may have the same volume or the volume may vary across the microfluidic device. The chambers may have a volume of less than or equal to about 1000 picoliters (pL), 900 pL, 800 pL, 700 pL, 600 pL, 500 pL, 400 pL, 300 pL, 200 pL, 100 pL, 75 pL, 50 pL, 25 pL, or less picoliters. The chambers may have a volume between about 25 pL and 50 pL, 25 pL and 75 pL, 25 pL and 100 pL, 25 pL and 200 pl, 25 pL and 300 pL, 25 pL and 400 pL, 25 pL and 500 pL, 25 pL and 600 pL, 25 pL and 700 pL, 25 pL and 800 pL, 25 pL and 900 pl, or 25 pL and 1000 pL. In an example, the chambers have a volume of less than or equal to 250 pL. In another example, the chambers have a volume of less than or equal to 150 pL. In another example, the chambers have a volume of approximately 100 pL.

In some embodiments, the lengths of the siphon apertures are constant. In some embodiments, the lengths of the siphon apertures vary. The siphon apertures may have a long dimension that is less than or equal to about 150 μm, less than or equal to about 100 μm, less than or equal to about 50 μm, less than or equal to about 25 μm, less than or equal to about 10 μm, less than or equal to about 5 μm, or less. In some embodiments, the depth of the siphon aperture may be less than or equal to about 50 μm, less than or equal to about 25 μm, less than or equal to about 10 μm, less than or equal to about 5 μm or less. The siphon apertures may have a cross-sectional width less than or equal to about 50 μm, less than or equal to about 40 μm, less than or equal to about 30 μm, less than or equal to about 20 μm, less than or equal to about 10 μm, less than or equal to about 5 μm, or less.

In some examples, the cross-sectional dimensions of the siphon aperture may be about 50 μm wide by about 50 μm deep. In some examples, the cross-sectional dimensions of the siphon aperture may be about 50 μm wide by about 40 μm deep. In some examples, the cross-sectional dimensions of the siphon aperture may be about 50 μm wide by about 30 μm deep. In some examples, the cross-sectional dimensions of the siphon aperture may be about 50 μm wide by about 20 μm deep. In some examples, the cross-sectional dimensions of the siphon aperture may be about 50 μm wide by about 10 μm deep. In some examples, the cross-sectional dimensions of the siphon aperture may be about 50 μm wide by about 5 μm deep. In some examples, the cross-sectional dimensions of the siphon aperture may be about 40 μm wide by about 50 μm deep. In some examples, the cross-sectional dimensions of the siphon aperture may be about 30 μm wide by about 50 μm deep. In some examples, the cross-sectional dimensions of the siphon aperture may be about 20 μm wide by about 50 μm deep. In some examples, the cross-sectional dimensions of the siphon aperture may be about 10 μm wide by about 50 μm deep. In some examples, the cross-sectional dimensions of the siphon aperture may be about 5 μm wide by about 50 μm deep. In some examples, the cross-sectional dimensions of the siphon aperture may be about 40 μm wide by about 40 μm deep. In some examples, the cross-sectional dimensions of the siphon aperture may be about 30 μm wide by about 30 μm deep. In some examples, the cross-sectional dimensions of the siphon aperture may be about 20 μm wide by about 20 μm deep. In some examples, the cross-sectional dimensions of the siphon aperture may be about 10 μm wide by about 10 μm deep. In some examples, the cross-sectional dimensions of the siphon aperture may be about 5 μm wide by about 5 μm deep. The cross-sectional shape of the siphon aperture may be any suitable cross-sectional shape including, but not limited to, circular, oval, triangular, square, or rectangular. In some embodiments, the cross-sectional area of the siphon aperture may be constant along the length of the siphon aperture. Alternatively, or in addition to, the cross-sectional area of the siphon aperture may vary along the length of the siphon aperture. The cross-sectional area of the siphon aperture may be greater at the connection to the channel than the cross-sectional area of the siphon aperture at the connection to the chamber. Alternatively, the cross-sectional area of the siphon aperture at the connection to the chamber may be greater than the cross-sectional area of the siphon aperture at the connection to the channel. The cross-sectional area of the siphon aperture may vary between about 50% and 150%, between about 60% and 125%, between about 70% and 120%, between about 80% and 115%, between about 90% and 110%, between about 95% and 100%, or between about 98% and 102%. The cross-sectional area of the siphon aperture may be less than or equal to about 2,500 μm², less than or equal to about 1,000 μm², less than or equal to about 750 μm², less than or equal to about 500 μm², less than or equal to about 250 μm², less than or equal to about 100 μm², less than or equal to about 75 μm², less than or equal to about 50 μm², less than or equal to about 25 μm², or less. The cross-sectional area of the siphon aperture at the connection to the channel may be less than or equal to the cross-sectional area of the channel. The cross-sectional area of the siphon aperture at the connection to the channel may be less than or equal to about 98%, less than or equal to about 95%, less than or equal to about 90%, less than or equal to about 85%, less than or equal to about 80%, less than or equal to about 75%, less than or equal to about 70%, less than or equal to about 60%, less than or equal to about 50%, less than or equal to about 40%, less than or equal to about 30%, less than or equal to about 20%, less than or equal to about 10%, less than or equal to about 5%, less than or equal to about 1%, or less than or equal to about 0.5% of the cross-sectional area of the channel.

In some embodiments, the siphon apertures are substantially perpendicular to the channel. In some embodiments, the siphon apertures are not substantially perpendicular to the channel. In some embodiments, an angle between the siphon apertures and the channel may be at least about 5°, at least about 10°, at least about 15°, at least about 20°, at least about 30°, at least about 40°, at least about 50°, at least about 60°, at least about 70°, at least about 90°, at least about 100°, at least about 110°, at least about 120°, at least about 130°, at least about 140°, at least about 150°, at least about 160°, or at least about 170°.

The microfluidic device may be configured to permit pressurized off-gassing or degassing of the channel, chamber, siphon aperture, or any combination thereof. Pressurized off-gassing or degassing may be provided by a film or membrane configured to permit pressurized off-gassing or degassing. The film or membrane may be permeable to gas above a pressure threshold. The film or membrane may not be permeable to (e.g., is impermeable or substantially impermeable to) liquids such as, but not limited to, aqueous fluids, oils, or other solvents. The channel, the chamber, the siphon aperture, or any combination thereof may comprise the film or membrane. In an example, the chamber comprises the gas permeable film or membrane and the channel and/or siphon aperture does not comprise the gas permeable film or membrane. In another example, the chamber and siphon aperture comprises the gas permeable film or membrane and the channel does not comprise the gas permeable film or membrane. In another example, the chamber, channel, and siphon aperture comprise the gas permeable film or membrane.

The film or membrane may be a thin file. The film or membrane may be a polymer. The film may be a thermoplastic film or membrane. The film or membrane may not comprise an elastomeric material. The gas permeable film or membrane may cover the fluid flow path, the channel, the chamber, or any combination thereof. In an example, the gas permeable film or membrane covers the chamber. In another example, the gas permeable film or membrane covers the chamber and the channel. The gas permeability of the film may be induced by elevated pressures. The thickness of the film or membrane may be less than or equal to about 500 micrometers (μm), 250 μm, 200 μm, 150 μm, 100 μm, 75 μm, 50 μm, 25 μm, or less. In an example, the film or membrane has a thickness of less than or equal to about 100 μm. In another example, the film or membrane has a thickness of less than or equal to about 50 μm. In another example, the film or membrane has a thickness of less than or equal to about 25 μm. The thickness of the film or membrane may be from about 0.1 μm to about 200 μm, 0.5 μm to 150 μm, or 25 μm to 100 μm. In an example, the thickness of the film or membrane is from about 25 μm to 100 μm. The thickness of the film may be selected by manufacturability of the film, the air permeability of the film, the volume of each chamber or partition to be out-gassed, the available pressure, and/or the time to complete the partitioning or digitizing process.

The film or membrane may be configured to employee different permeability characteristics under different applied pressure differentials. For example, the thin film may be gas impermeable at a first pressure differential (e.g., low pressure) and at least partially gas permeable at a second pressure differential (e.g., high pressure). The first pressure differential (e.g., low pressure differential) may be less than or equal to about 8 pounds per square inch (psi), 6 psi, 4 psi, 2 psi, 1 psi, or less. In an example, the film or membrane is substantially impermeable to gas at a pressure differential of less than 4 psi. The second pressure differential (e.g., high pressure differential) may be greater than or equal to about 1 psi, 2 psi, 4 psi, 6 psi, 8 psi, 10 psi, 12 psi 14 psi, 16 psi, 20 psi, or more. In an example, the film or membrane is substantially gas permeable at a pressure of greater than or equal to 4 psi.

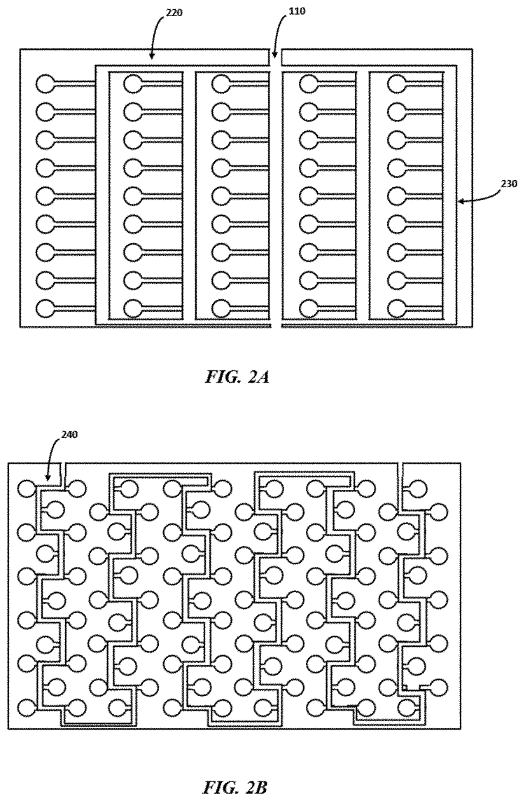

The chambers may be arranged in a variety of patterns. FIGS. 2A and 2B illustrate example patterns of chamber, siphon aperture, and channel arrangements. In some embodiments, multiple channels are employed, while in some embodiments, a single channel may be used. In some embodiments, a channel may comprise a group of sub-channels. The group of sub-channels may be connected by one or more cross-channels. In some of these embodiments, the sub-channels are substantially parallel to one another so that the array of chambers forms a grid of chambers. FIG. 2A illustrates an embodiment in which parallel sub-channels 230 and one or more cross-channels 220 are used to form a grid of chambers.

In some embodiments, chambers are constructed so as to form a hexagonal grid of chambers, with curved or angled sub-channels connecting the chambers. A hexagonal grid of chambers may also be formed and connected by a single channel, such as by a channel that forms a serpentine pattern 240 across the microfluidic device. FIG. 2B illustrates an embodiment in which a single channel in a serpentine pattern forms a hexagonal grid of chambers.

In some embodiments, the lengths of the sub-channels are constant. In some embodiments, the lengths of the sub-channel may vary. The sub-channel may have a long dimension that is less than or equal to 100 mm, less than or equal to about 75 mm, less than or equal to about 50 mm, less than or equal to about 40 mm, less than or equal to about 30 mm, less than or equal to about 20 mm, less than or equal to about 10 mm, less than or equal to about 8 mm, less than or equal to about 6 mm, less than or equal to about 4 mm, less than or equal to about 2 mm, or less. The length of the sub-channel may be bounded by the external length or width of the microfluidic device. In some embodiments, the sub-channel may have the same cross-sectional dimension as the channel. In some embodiments, the sub-channel may have different cross-sectional dimension than the channel. In some embodiments, the sub-channel may have the same depth as the channel and a different cross-sectional dimension. In some embodiments, the sub-channel may have the same cross-sectional dimension as the channel and a different depth. For example, the sub-channel may have a depth of less than or equal to about 500 μm, less than or equal to about 250 μm, less than or equal to about 100 μm, less than or equal to about 80 μm, less than or equal to about 60 μm, less than or equal to about 30 μm, less than or equal to about 15 μm, or less. The sub-channel may have a cross-section width of less than or equal to about 500 μm, less than or equal to about 250 μm, less than or equal to about 100 μm, less than or equal to about 75 μm, less than or equal to about 50 μm, less than or equal to about 40 μm, less than or equal to about 30 μm, less than or equal to about 20 μm, less than or equal to about 10 μm, or less.

In some examples, the cross-sectional dimensions of the sub-channel may be about 100 μm wide by about 100 μm deep. In some examples, the cross-sectional dimensions of the sub-channel may be about 100 μm wide by about 80 μm deep. In some examples, the cross-sectional dimensions of the sub-channel may be about 100 μm wide by about 60 μm deep. In some examples, the cross-sectional dimensions of the sub-channel may be about 100 μm wide by about 40 μm deep. In some examples, the cross-sectional dimensions of the sub-channel may be about 100 μm wide by about 20 μm deep. In some examples, the cross-sectional dimensions of the sub-channel may be about 100 μm wide by about 10 μm deep. In some examples, the cross-sectional dimensions of the sub-channel may be about 80 μm wide by about 100 μm deep. In some examples, the cross-sectional dimensions of the sub-channel may be about 60 μm wide by about 100 μm deep. In some examples, the cross-sectional dimensions of the sub-channel may be about 40 μm wide by about 100 μm deep. In some examples, the cross-sectional dimensions of the sub-channel may be about 20 μm wide by about 100 μm deep. In some examples, the cross-sectional dimensions of the sub-channel may be about 10 μm wide by about 100 μm deep. In some examples, the cross-sectional dimensions of the sub-channel may be about 80 μm wide by about 80 μm deep. In some examples, the cross-sectional dimensions of the sub-channel may be about 60 μm wide by about 60 μm deep. In some examples, the cross-sectional dimensions of the sub-channel may be about 40 μm wide by about 40 μm deep. In some examples, the cross-sectional dimensions of the sub-channel may be about 20 μm wide by about 20 μm deep. In some examples, the cross-sectional dimensions of the sub-channel may be about 10 μm wide by about 10 μm deep. The cross-sectional shape of the sub-channel may be any suitable cross-sectional shape including, but not limited to, circular, oval, triangular, square, or rectangular. In some embodiments, the cross sectional shape of the sub-channel is different than the cross-sectional shape of the channel. In some embodiments, the cross-sectional shape of the sub-channel is the same as the cross-sectional shape of the channel. The cross-sectional area of the sub-channel may be constant along the length of the sub-channel. Alternatively, or in addition to, the cross-sectional area of the sub-channel may vary along the length of the channel. The cross-sectional area of the sub-channel may vary between about 50% and 150%, between about 60% and 125%, between about 70% and 120%, between about 80% and 115%, between about 90% and 110%, between about 95% and 100%, or between about 98% and 102%. The cross-sectional area of the sub-channel may be less than or equal to about 10,000 μm², less than or equal to about 7,500 μm², less than or equal to about 5,000 μm², less than or equal to about 2,500 μm², less than or equal to about 1,000 μm², less than or equal to about 750 μm², less than or equal to about 500 μm², less than or equal to about 400 μm², less than or equal to about 300 μm², less than or equal to about 200 μm², less than or equal to about 100 μm², or less. In some embodiments, the cross-sectional area of the sub-channel is the same as the cross-sectional area of the channel. In some embodiments, the cross-sectional area of the sub-channel may be less than or equal to the area of the cross-sectional area of the channel. The cross-sectional area of the sub-channel may be less than or equal to about 98%, less than or equal to about 95%, less than or equal to about 90%, less than or equal to about 85%, less than or equal to about 80%, less than or equal to about 75%, less than or equal to about 70%, less than or equal to about 60%, less than or equal to about 50%, less than or equal to about 40%, less than or equal to about 30%, less than or equal to about 20%, less than or equal to about 20%, or less of the cross-sectional area of the channel.

In some embodiments, the lengths of the cross-channels are constant. In some embodiments, the lengths of the cross-channel may vary. The cross-channel may have a long dimension that is less than or equal to about 100 mm, less than or equal to about 75 mm, less than or equal to about 50 mm, less than or equal to about 40 mm, less than or equal to about 30 mm, less than or equal to about 20 mm, less than or equal to about 10 mm, less than or equal to about 8 mm, less than or equal to about 6 mm, less than or equal to about 4 mm, less than or equal to about 2 mm, or less. The length of the cross-channel may be bounded by the external length or width of the microfluidic device. In some embodiments, the cross-channel may have the same cross-sectional dimension as the channel. In some embodiments, the cross-channel may have a different cross-sectional dimension than the channel. In some embodiments, the cross-channel may have the same depth as the channel and a different cross-sectional dimension. In some embodiments, the cross-channel may have the same cross-sectional dimension as the channel and a different depth. For example, the cross-channel may have a depth of less than or equal to about 500 μm, less than or equal to about 250 μm, less than or equal to about 100 μm, less than or equal to about 80 μm, less than or equal to about 60 μm, less than or equal to about 30 μm, less than or equal to about 15 μm, or less. The cross-channel may have a cross-section width of less than or equal to about 500 μm, less than or equal to about 250 μm, less than or equal to about 100 μm, less than or equal to about 75 μm, less than or equal to about 50 μm, less than or equal to about 40 μm, less than or equal to about 30 μm, less than or equal to about 20 μm, less than or equal to about 10 μm, or less.

In some examples, the cross-sectional dimensions of the cross-channel may be about 100 μm wide by about 100 μm deep. In some examples, the cross-sectional dimensions of the cross-channel may be about 100 μm wide by about 80 μm deep. In some examples, the cross-sectional dimensions of the cross-channel may be about 100 μm wide by about 60 μm deep. In some examples, the cross-sectional dimensions of the cross-channel may be about 100 μm wide by about 40 μm deep. In some examples, the cross-sectional dimensions of the cross-channel may be about 100 μm wide by about 20 μm deep. In some examples, the cross-sectional dimensions of the cross-channel may be about 100 μm wide by about 10 μm deep. In some examples, the cross-sectional dimensions of the cross-channel may be about 80 μm wide by about 100 μm deep. In some examples, the cross-sectional dimensions of the cross-channel may be about 60 μm wide by about 100 μm deep. In some examples, the cross-sectional dimensions of the cross-channel may be about 40 μm wide by about 100 μm deep. In some examples, the cross-sectional dimensions of the cross-channel may be about 20 μm wide by about 100 μm deep. In some examples, the cross-sectional dimensions of the cross-channel may be about 10 μm wide by about 100 μm deep. In some examples, the cross-sectional dimensions of the cross-channel may be about 80 μm wide by about 80 μm deep. In some examples, the cross-sectional dimensions of the cross-channel may be about 60 μm wide by about 60 μm deep. In some examples, the cross-sectional dimensions of the cross-channel may be about 40 μm wide by about 40 μm deep. In some examples, the cross-sectional dimensions of the cross-channel may be about 20 μm wide by about 20 μm deep. In some examples, the cross-sectional dimensions of the cross-channel may be about 10 μm wide by about 10 μm deep.

The cross-sectional shape of the cross-channel may be any suitable cross-sectional shape including, but not limited to, circular, oval, triangular, square, or rectangular. In some embodiments, the cross sectional shape of the cross-channel is different than the cross-sectional shape of the channel. In some embodiments, the cross-sectional shape of the cross-channel is the same as the cross-sectional shape of the channel. The cross-sectional area of the cross-channel may be constant down the length of the cross-channel. Alternatively, or in addition to, the cross-sectional area of the cross-channel may vary down the length of the channel. The cross-sectional area of the cross-channel may vary between about 50% and 150%, between about 60% and 125%, between about 70% and 120%, between about 80% and 115%, between about 90% and 110%, between about 95% and 100%, or between about 98% and 102%. The cross-sectional area of the cross-channel may be less than or equal to about 10,000 μm², less than or equal to about 7,500 μm², less than or equal to about 5,000 μm², less than or equal to about 2,500 μm², less than or equal to about 1,000 μm², less than or equal to about 750 μm², less than or equal to about 500 μm², less than or equal to about 400 μm², less than or equal to about 300 μm², less than or equal to about 200 μm², less than or equal to about 100 μm², or less. In some embodiments, the cross-sectional area of the cross-channel is the same as the cross-sectional area of the channel. In some embodiments, the cross-sectional area of the cross-channel is less than the area of the cross-sectional area of the channel. The cross-sectional area of cross-channel may be less than or equal to about 98%, less than or equal to about 95%, less than or equal to about 90%, less than or equal to about 85%, less than or equal to about 80%, less than or equal to about 75%, less than or equal to about 70%, less than or equal to about 60%, less than or equal to about 50%, less than or equal to about 40%, less than or equal to about 30%, less than or equal to about 20%, less than or equal to about 20%, or less of the cross-sectional area of the channel.

### Method for Fabricating a Microfluidic Device

In an aspect, the present disclosure provides methods for fabricating a microfluidic device. The method may comprise injection molding a thermoplastic to create a microfluidic structure. The microfluidic structure may comprise a channel, a plurality of chambers, and a plurality of siphon apertures. The plurality of chambers may be connected to the channel by the plurality of siphon apertures. The channel may comprise an inlet and an outlet. A thermoplastic thin film may be applied to cap the microfluidic structure. The thermoplastic thin film may be at least partially gas permeable when a pressure differential is applied across the thermoplastic thin film.

In some embodiments, the thermoplastic thin film is formed by injection molding. The thermoplastic thin film may be applied to the microfluidic structure by thermal bonding. Alternatively, or in addition to, the thin film may be applied by chemical bonding. In some embodiments, the thermoplastic thin film is formed as part of and during the injection molding process to form the microfluidic device.

The body of the microfluidic device and the thin film may comprise the same materials. Alternatively, the body of the microfluidic device and the thin film may comprise different materials. The body of the microfluidic device and the thin film may comprise a thermoplastic. Example thermoplastics include, but are not limited to, cyclo-olefin polymers, acrylic, acrylonitrile butadiene styrene, nylon, polylactic acid, polybenzimidazole, polycarbonate, polyether sulfone, poly ether ether ketone, polyetherimide, polyethylene, polyphenylene oxide, polyphenylene sulfide, polypropylene, polystyrene, polyvinyl chloride, polytetrafluoroethylene, polyester, polyurethane or any derivative thereof. The microfluidic device may comprise homopolymers, copolymers, or a combination thereof. The microfluidic device may be formed of inelastic materials. Alternatively or in addition to, the microfluidic device may be formed of elastic materials.

In an example embodiment of the present disclosure, both the thermoplastic and the thin film are composed of a cyclo-olefin polymer. One suitable thermoplastic is Zeonor 1430R (Zeon Chemical, Japan) while one suitable thin film is Zeonox 1060R (Zeon Chemical, Japan). In some embodiments, the thin film is a material that is gas-impermeable at low pressure and at least partially gas permeable under pressure.

In some embodiments, the inlet and the outlet are formed by mechanical drilling. In some embodiments, the inlet and outlet are formed by melting, dissolving, or etching the thermoplastic.

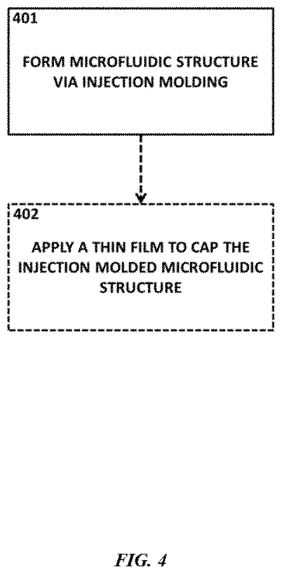

FIG. 4 illustrates a method of manufacture of embodiments of the present disclosure. In FIG. 4, an injection molding process 401 is used to form a microfluidic structure. The microfluidic structure includes an array of chambers, which are connected to at least one channel via siphon apertures, as shown in FIGS. 1A and IB. The microfluidic structure is capped by a thin film. In the capping process, openings in at least one side of the microstructure are covered over in order to close and seal the microstructures. In some embodiments of the present disclosure, the capping is performed by a process 402 of applying a thin film to the injection molded microfluidic structure. In some embodiments of the present disclosure, the capping is performed by forming the thin film as part of the injection molding process 401.

As another example, while described in the context of a microstructure which is formed via injection molding, microfluidic devices formed by other microfabrication techniques may also benefit from the use of such a thin thermoplastic film to allow outgassing as described above. Such techniques include micromachining, microlithography, and hot embossing, as well as other microfabrication techniques.

### Method of Analyzing a Nucleic Acid Sample

In an aspect, the present disclosure provides method for thermal cycling a microfluidic device. The method may comprise providing a microfluidic device in fluid communication with a pneumatic module and thermal communication with a thermal module. The microfluidic device may comprise a plurality of chambers. A surface of the microfluidic device may comprise a film or barrier. The film or barrier may be in thermal communication with the thermal module. The pneumatic module may be used to load a nucleic acid sample comprising at least one nucleic acid molecule into a chamber of the plurality of chambers. The pneumatic module may additionally be used to apply pressure to the microfluidic device to provide and maintain thermal contact between the film or barrier and the thermal module. The thermal module may be used to thermal cycle the plurality of chambers to amplify the at least one nucleic acid molecule in the chamber. A single round of thermal cycling may be completed within about 20 second or less. The thermal module may maintain a temperature across the plurality of chambers within 0.2° C. The method may further comprise using an optical module to image the plurality of chambers. The optical module may be used to detect a presence or absence of the nucleic acid molecule in a chamber of the plurality of chambers.

In another aspect, the present disclosure provides method for thermal cycling a microfluidic device. The method may comprise providing a microfluidic device in fluid communication with a pneumatic module and thermal communication with a thermal module. The microfluidic device may comprise a plurality of chambers. A surface of the microfluidic device may comprise a film or barrier. The film or barrier may have a thermal conductivity that is less than or equal to about 1 watt per meter Kelvin (W/m-K). The film or barrier may be in thermal communication with the thermal module. The pneumatic module may be used to load a nucleic acid sample comprising at least one nucleic acid molecule into a chamber of the plurality of chambers. The pneumatic module may additionally be used to apply pressure to the microfluidic device to provide and maintain thermal contact between the film or barrier and the thermal module. The thermal module may be used to thermal cycle the plurality of chambers to amplify the at least one nucleic acid molecule in the chamber. A single round of thermal cycling may be completed within about 20 second or less. The method may further comprise using an optical module to image the plurality of chambers. The optical module may be used to detect a presence or absence of the nucleic acid molecule in a chamber of the plurality of chambers.

In another aspect, the present disclosure provides methods for using a microfluidic device to analyze a nucleic acid sample. The method may comprise providing a microfluidic device comprising a channel. The channel may comprise an inlet and an outlet. The microfluidic device may further comprise a plurality of chambers connected to the channel by a plurality of siphon apertures. The microfluidic device may be sealed by a thermoplastic thin film disposed adjacent to a surface of the microfluidic device such that the thermoplastic thin film caps the channel, the plurality of chambers, and the plurality of siphon apertures. A reagent may be applied to the inlet or the outlet. The microfluidic device may be filled by providing a first pressure differential between the reagent and the microfluidic device, causing the reagent to flow into the microfluidic device. The reagent may be partitioned into the chambers by applying a second pressure differential between the channel and the plurality of chambers to move the reagent into the plurality of chambers and to force gas within the plurality of chambers to pass through the thermoplastic thin film. The second pressure differential may be greater than the first pressure differential. A third pressure differential between the inlet and the outlet may be applied to introduce a fluid into the channel without introducing the fluid into the chambers. The third pressure differential may be less than the second pressure differential.

In another aspect, the present disclosure may provide methods for rapidly thermal cycling a microfluidic device. The method may comprise providing a microfluidic device comprising a plurality of chambers, contacting at least one surface of the microfluidic device with a thermal module (e.g., thermal cycler), using a fluid flow module (e.g., a pneumatic manifold or pneumatic module) to apply downward pressure to the microfluidic device, and using the thermal module to thermal cycle the microfluidic device. The plurality of chambers may be in thermal communication with the thermal module (e.g., thermal cycler). The microfluidic device may be disposed between the thermal module and the fluid flow module (e.g., pneumatic manifold or pneumatic module). The microfluidic device may be sandwiched between the thermal module and the fluid flow module (e.g., pneumatic manifold or pneumatic module). The pressure applied by the fluid flow module (e.g., pneumatic manifold or pneumatic module) may reduce or prevent the microfluidic device from warping or bending during thermal cycling. The pressure applied by the fluid flow module (e.g., pneumatic manifold or pneumatic module) may permit the microfluidic device to remain in contact with the thermal module during thermal cycling. The thermal cycling may activate or control a polymerase chain reaction.

The pneumatic manifold or module may apply a pressure across the entire microfluidic device. Alternatively, the pneumatic manifold or module may apply a pressure along one or more sides of the microfluidic device to prevent warping. In an example, the pneumatic manifold or module applies a pressure along the sides of the microfluidic device such that the center of the microfluidic device is not contacted by the pneumatic manifold or module. The portion of the microfluidic device not contacted by the pneumatic manifold of module may be optically available for imaging during processing of the microfluidic device. The pneumatic manifold or module may be movable. The movable pneumatic manifold or module may be in an engaged state in which the pneumatic manifold or module is in fluid communication with the microfluidic device. Alternatively, the pneumatic manifold or module may be in a non-engaged state in which the pneumatic manifold or module is not in fluid communication with the microfluidic device. When the pneumatic manifold or module is in the engaged state (e.g., in fluid communication with the microfluidic device) the pneumatic manifold may be locked in place using, for example, a mechanical or electrical locking mechanism. The microfluidic device may be disposed on or in contact with a piston pump or other positive displacement pump. The piston pump or other positive displacement pump may push the microfluidic device against the pneumatic manifold or module. In an example, the thermal cycle is on a piston pump and the piston pump applies a pressure (e.g., upward force) to contact the microfluidic device with the thermal module (e.g., thermal cycler) and the pneumatic manifold or module to sandwich the microfluidic device between the thermal module (e.g., thermal cycler) and the pneumatic manifold or module.

Rapid sample containers of the present disclosure may permit rapid heating and cooling, such as at heating or cooling rates of at least about 1° C./s, 5° C./s, 10° C./s, 15° C./s, 20° C./s, or greater. Such rapid heating and cooling rates may be achieved, for example, using material with substantially low thermal mass, such as using a sample container that is formed of one or more polymeric materials, in some cases without other types of materials (e.g., a container formed of only one or more polymeric materials).

During use, such sample container may be used to conduct a nucleic amplification reaction on a nucleic acid sample (e.g., DNA), such as PCR. This may include subjecting the nucleic acid sample to one or more cycles, each cycle comprising denaturation conditions (e.g., denaturation temperature or temperature range, and denaturation time) and elongation conditions (e.g., elongation temperature or temperature range, and elongation time). As an alternative, nucleic acid amplification may be performed isothermally.

In some embodiments, the inlet and the outlet are in fluid communication with a pneumatic pump. In some embodiments, the microfluidic device is in contact with a vacuum system. Filling and partitioning of a sample may be performed by applying pressure differentials across various features of the microfluidic device. In some embodiments, filling and partitioning of the sample may be performed without the use of valves between the chambers and the channel to isolate the sample. For example, filling of the channel may be performed by applying a pressure differential between the sample to be loaded and the channel. This pressure differential may be achieved by pressurizing the sample or by applying vacuum to the channel. Filling the chambers may be performed by applying a pressure differential between the channel and the chambers. This may be achieved by pressurizing the channel or by applying a vacuum to the chambers. Partitioning the sample may be performed by applying a pressure differential between a fluid and the channel. This pressure differential may be achieved by pressurizing the fluid or by applying a vacuum to the channel.

The thin film may employee different permeability characteristics under different applied pressure differentials. For example, the thin film may be gas impermeable at the first and third pressure differentials (e.g., low pressure), which may be smaller magnitude pressure differentials. The thin film may be at least partially gas permeable at the second pressure differential (e.g., high pressure), which may be a higher magnitude pressure differential. The first and third pressure differentials may be the same or they may be different. The first pressure differential may be the difference in pressure between the reagent in the inlet or outlet and the microfluidic device. During filling of the microfluidic device, the pressure of the reagent may be higher than the pressure of the microfluidic device. During filling of the microfluidic device, the pressure difference between the reagent and the microfluidic device (e.g., low pressure) may be less than or equal to about 8 pounds per square inch (psi), less than or equal to about 6 psi, less than or equal to about 4 psi, less than or equal to about 2 psi, less than or equal to about 1 psi, or less. In some examples, during filling of the microfluidic device, the pressure differential between the reagent and the microfluidic device may be from about 1 psi to about 8 psi. In some examples, during filling of the microfluidic device, the pressure differential between the reagent and the microfluidic device may be from about 1 psi to about 6 psi. In some examples, during filling of the microfluidic device, the pressure differential between the reagent and the microfluidic device may be from about 1 psi to about 4 psi. The microfluidic device may be filled by applying a pressure differential between the reagent and the microfluidic device for less than or equal to about 20 minutes, less than or equal to about 15 minutes, less than or equal to about 10 minutes, less than or equal to about 5 minutes, less than or equal to about 3 minutes, less than or equal to about 2 minutes, less than or equal about 1 minute, or less.

A filled microfluidic device may have reagent in the channel, siphon apertures, chambers, or any combination thereof. Backfilling of the reagent into the chambers may occur upon filling of the microfluidic device or may occur during application of a second pressure differential. The second pressure differential (e.g., high pressure) may correspond to the difference in pressure between the channel and the plurality of chambers. During application of the second pressure differential a first fluid in the higher pressure domain may push a second fluid in the lower pressure domain through the thin film and out of the microfluidic device. The first and second fluids may comprise a liquid or a gas. The liquid may comprise an aqueous mixture or an oil mixture. The second pressure differential may be achieved by pressurizing the channel. Alternatively, or in addition to, the second pressure differentially may be achieved by applying a vacuum to the chambers. During application of the second pressure differential, reagent in the channel may flow into the chambers. Additionally, during the application of the second pressure differential gas trapped within the siphon apertures, chambers, and channel may outgas through the thin film. During backfilling and outgassing of the chambers, the pressure differential between the chambers and the channel may be greater than or equal to about 6 psi, greater than or equal to about 8 psi, greater than or equal to about 10 psi, greater than or equal to about 12 psi, greater than or equal to about 14 psi, greater than or equal to about 16 psi, greater than or equal to about 18 psi, greater than or equal to about 20 psi, or greater. In some examples, during backfilling of the chambers, the pressure differential between the chambers and the channel is from about 8 psi to about 20 psi. In some examples, during backfilling of the chambers, the pressure differential between the chambers and the channel is from about 8 psi to about 18 psi. In some examples, during backfilling of the chambers, the pressure differential between the chambers and the channel is from about 8 psi to about 16 psi. In some examples, during backfilling of the chambers, the pressure differential between the chambers and the channel is from about 8 psi to about 14 psi. In some examples, during backfilling of the chambers, the pressure differential between the chambers and the channel is from about 8 psi to about 12 psi. In some examples, during backfilling of the chambers, the pressure differential between the chambers and the channel is from about 8 psi to about 10 psi. The chambers may be backfilled and outgassed by applying a pressure differential for more than about 5 minutes, more than about 10 minutes, more than about 15 minutes, more than about 20 minutes, more than about 25 minutes, more than about 30 minutes, or more.

The sample may be partitioned by removing the excess sample from the channel. Removing excess sample from the channel may prevent reagents in one chamber from diffusing through the siphon aperture into the channel and into other chambers. Excess sample within the channel may be removed by introducing a fluid to the inlet or the outlet of the channel. The pressure of the fluid may be greater than the pressure of the channel, thereby creating a pressure differential between the fluid and the channel. The fluid may be oxygen, nitrogen, carbon dioxide, air, a noble gas, or any combination thereof. During partitioning of the sample, the pressure differential between the fluid and the channel may be less than or equal to about 8 psi, less than or equal to about 6 psi, less than or equal to about 4 psi, less than or equal to about 2 psi, less than or equal to about 1 psi, or less. In some examples, during partitioning of the sample, the pressure differential between the fluid and the channel may be from about 1 psi to about 8 psi. In some examples, during partitioning of the sample, the pressure differential between the fluid and the channel may be from about 1 psi to about 6 psi. In some examples, during partitioning of the sample, the pressure differential between the fluid and the channel may be from about 1 psi to about 4 psi. The sample may be partitioned by applying a pressure differential between the fluid and the channel for less than or equal to about 20 minutes, less than or equal to about 15 minutes, less than or equal to about 10 minutes, less than or equal to about 5 minutes, less than or equal to about 3 minutes, less than or equal to about 2 minutes, less than or equal to about 1 minute, or less.

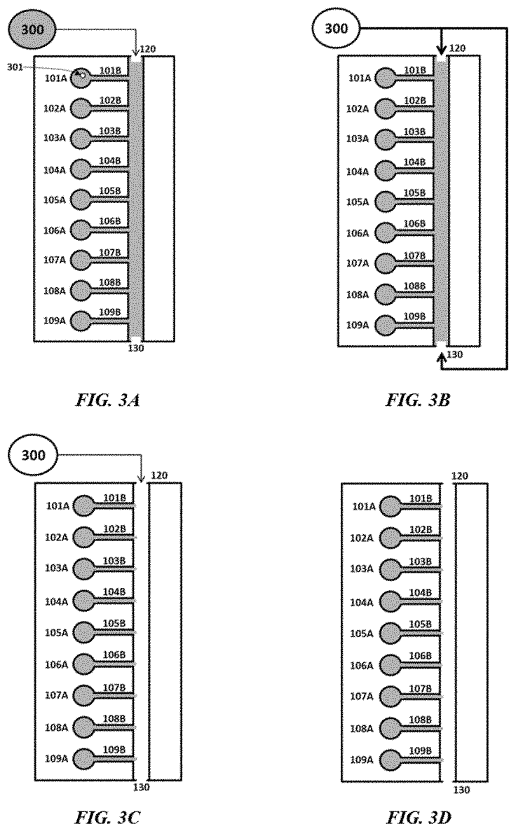

FIGS. 3A-3D illustrate a method for use of the microfluidic device shown in FIG. 1A. In FIG. 3A, a low pressure is applied to reagent at the inlet 120 via a pneumatic pump 300 to force reagent into the channel 110 and thereby fill the chambers via the siphon apertures. The pressure forces reagent to flow through the channel, and thereby to flow into the chambers via the siphon apertures. At this time, gas bubbles such as bubble 301 may remain within the chambers, siphon apertures, or channel. Filling via the application of low pressure may continue until the chambers, siphon apertures, and channel are substantially filled with reagent. The reagent may be a reagent to be used in a polymerase chain reaction. In some embodiments, the reagent is diluted such that no more than one PCR template is present in the reagent per chamber of the microfluidic device.

In FIG. 3B, the pneumatic pump 300 is connected to both inlets 120 and outlets 130 and a high pressure is applied. The high pressure is transmitted via the reagent and applied to gas bubbles such as bubble 301. Under the influence of this high pressure, thin film 150 becomes gas permeable, and the bubble 301 can outgas through the thin film 150. By applying this high pressure, the chambers, siphon apertures, and channel can be rendered substantially free of gas bubbles, thereby avoiding fouling.

In FIG. 3C, fluid is reintroduced by applying low pressure to a gas at the inlet 120 via pneumatic pump 300. The air pressure may not be sufficient to allow the gas to outgas through the thin film or high enough to force gas bubbles into the siphon apertures and chambers. Instead, the gas may clear the channel of reagent, leaving the reagent isolated in each chamber and siphon aperture. In some embodiments, the gas is air. In some embodiments, the gas may be an inert gas such as nitrogen, carbon dioxide, or a noble gas. Such a gas may be used to avoid reaction between the reagent and the component gases of air.

FIG. 3D illustrates the state of the system after the low pressure has been applied in FIG. 3C. After application of the low pressure gas the chambers and siphon apertures may remain filled with reagent, while the channel may be cleared of reagent. The reagent may remain stationary within the chambers due to the capillary force and high surface tension created by the siphon aperture. The capillary force and high surface tension may prevent the reagent from flowing into the channel and minimize reagent evaporation.

Partitioning of the sample may be verified by the presence of an indicator within the reagent. An indicator may include a molecule comprising a detectable moiety. The detectable moiety may include radioactive species, fluorescent labels, chemiluminescent labels, enzymatic labels, colorimetric labels, or any combination thereof. Non-limiting examples of radioactive species include ³H, ¹⁴C, ²²Na, ³²P, ³³P, ³⁵S, ⁴²K, ⁴⁵Ca, ⁵⁹Fe, ¹²³I, ¹²⁴I, ¹²⁵I, ¹³¹I, or ²⁰³Hg. Non-limiting examples of fluorescent labels include fluorescent proteins, optically active dyes (e.g., a fluorescent dye), organometallic fluorophores, or any combination thereof. Non-limiting examples of chemiluminescent labels include enzymes of the luciferase class such as Cypridina, Gaussia, Renilla, and Firefly luciferases. Non-limiting examples of enzymatic labels include horseradish peroxidase (HRP), alkaline phosphatase (AP), beta galactosidase, glucose oxidase, or other labels.

In some embodiments, an indicator molecule is a fluorescent molecule. Fluorescent molecules may include fluorescent proteins, fluorescent dyes, and organometallic fluorophores. In some embodiments, the indicator molecule is a protein fluorophore. Protein fluorophores may include green fluorescent proteins (GFPs, fluorescent proteins that fluoresce in the green region of the spectrum, generally emitting light having a wavelength from 500-550 nanometers), cyan-fluorescent proteins (CFPs, fluorescent proteins that fluoresce in the cyan region of the spectrum, generally emitting light having a wavelength from 450-500 nanometers), red fluorescent proteins (RFPs, fluorescent proteins that fluoresce in the red region of the spectrum, generally emitting light having a wavelength from 600-650 nanometers). Non-limiting examples of protein fluorophores include mutants and spectral variants of AcGFP, AcGFP1, AmCyan, AmCyanl, AQ143, AsRed2, Azami Green, Azurite, BFP, Cerulean, CFP, CGFP, Citrine, copGFP, CyPet, dKeima-Tandem, DsRed, dsRed-Express, DsRed-Monomer, DsRed2, dTomato, dTomato-Tandem, EBFP, EBFP2, ECFP, EGFP, Emerald, EosFP, EYFP, GFP, HcRed-Tandem, HcRedl, JRed, Katuska, Kusabira Orange, Kusabira Orange2, mApple, mBanana, mCerulean, mCFP, mCherry, mCitrine, mECFP, mEmerald, mGrape1, mGrape2, mHoneydew, Midori-Ishi Cyan, mKeima, mKO, mOrange, mOrange2, mPlum, mRaspberry, mRFP1, mRuby, mStrawberry, mTagBFP, mTangerine, mTeal, mTomato, mTurquoise, mWasabi, PhiYFP, ReAsH, Sapphire, Superfolder GFP, T-Sapphire, TagCFP, TagGFP, TagRFP, TagRFP-T, TagYFP, tdTomato, Topaz, TurboGFP, Venus, YFP, YPet, ZsGreen, and ZsYellowl.

In some embodiments, the indicator molecule is a fluorescent dye. Non-limiting examples of fluorescent dyes include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, phenanthridines and acridines, ethidium bromide, propidium iodide, hexidium iodide, dihydroethidium, ethidium homodimer-1 and -2, ethidium monoazide, and ACMA, Hoechst 33258, Hoechst 33342, Hoechst 34580, DAPI, acridine orange, 7-AAD, actinomycin D, LDS751, hydroxystilbamidine, SYTOX Blue, SYTOX Green, SYTOX Orange, POPO-1, POPO-3, YOYO-1, YOYO-3, TOTO-1, TOTO-3, JOJO-1, LOLO-1, BOBO-1, BOBO-3, PO-PRO-1, PO-PRO-3, BO-PRO-1, BO-PRO-3, TO-PRO-1, TO-PRO-3, TO-PRO-5, JO-PRO-1, LO-PRO-1, YO-PRO-1, YO-PRO-3, PicoGreen, OliGreen, RiboGreen, SYBR Gold, SYBR Green I, SYBR Green II, SYBR DX, SYTO-40, -41, -42, -43, -44, -45 (blue), SYTO-13, -16, -24, -21, -23, -12, -11, -20, -22, -15, -14, -25 (green), SYTO-81, -80, -82, -83, -84, -85 (orange), SYTO-64, -17, -59, -61, -62, -60, -63 (red), fluorescein, fluorescein isothiocyanate (FITC), tetramethyl rhodamine isothiocyanate (TRITC), rhodamine, tetramethyl rhodamine, R-phycoerythrin, Cy-2, Cy-3, Cy-3.5, Cy-5, Cy5.5, Cy-7, Texas Red, Phar-Red, allophycocyanin (APC), Sybr Green I, Sybr Green II, Sybr Gold, CellTracker Green, 7-AAD, ethidium homodimer I, ethidium homodimer II, ethidium homodimer III, ethidium bromide, umbelliferone, eosin, green fluorescent protein, erythrosin, coumarin, methyl coumarin, pyrene, malachite green, stilbene, lucifer yellow, cascade blue, dichlorotriazinylamine fluorescein, dansyl chloride, fluorescent lanthanide complexes such as those including europium and terbium, carboxy tetrachloro fluorescein, 5 and/or 6-carboxy fluorescein (FAM), 5- (or 6-) iodoacetamidofluorescein, 5-{[2(and 3)-5-(Acetylmercapto)-succinyl]amino} fluorescein (SAMSA-fluorescein), lissamine rhodamine B sulfonyl chloride, 5 and/or 6 carboxy rhodamine (ROX), 7-amino-methyl-coumarin, 7-Amino-4-methylcoumarin-3-acetic acid (AMCA), BODIPY fluorophores, 8-methoxypyrene-1,3,6-trisulfonic acid trisodium salt, 3,6-Disulfonate-4-amino-naphthalimide, phycobiliproteins, AlexaFluor 350, 405, 430, 488, 532, 546, 555, 568, 594, 610, 633, 635, 647, 660, 680, 700, 750, and 790 dyes, DyLight 350, 405, 488, 550, 594, 633, 650, 680, 755, and 800 dyes, or other fluorophores.

In some embodiments, the indicator molecule is an organometallic fluorophore. Non limiting examples of organometallic fluorophores include lanthanide ion chelates, nonlimiting examples of which include tris(dibenzoylmethane) mono(1,10-phenanthroline)europium(III), tris(dibenzoylmethane) mono(5-amino-1,10-phenanthroline)europium (III), and Lumi4-Tb cryptate.

In some embodiments, the images are taken of the microfluidic device. Images may be taken of single chambers, an array of chambers, or of multiple arrays of chambers concurrently. In some embodiments, the images are taken through the body of the microfluidic device. In some embodiments, images are taken through the thin film of the microfluidic device. In some embodiments, images are taken through both the body of the microfluidic device and through the thin film. In some embodiments, the body of the microfluidic device is substantially optically transparent. In some embodiments, the body of the microfluidic device is substantially optically opaque. In some embodiments, the thin film is substantially optically transparent. In some embodiments, images may be taken prior to filling the microfluidic device with reagent. In some embodiments, images may be taken after filling of the microfluidic device with reagent. In some embodiments, images may be taken during filling the microfluidic device with reagent. In some embodiments, images are taken to verify partitioning of the reagent. In some embodiments, images are taken during a reaction to monitor products of the reaction. In some embodiments, products of the reaction comprise amplification products. In some embodiments, images are taken at specified intervals. Alternatively, or in addition to, a video may be taken of the microfluidic device. The specified intervals may include taking an image at least every 300 seconds, at least every 240 seconds, at least every 180 seconds, at least every 120 seconds, at least every 90 seconds, at least every 60 seconds, at least every 30 seconds, at least every 15 seconds, at least every 10 seconds, at least every 5 seconds, at least every 4 seconds, at least every 3 seconds, at least every 2 seconds, at least every 1 second, or more frequently during a reaction.

In some embodiments, the method for using a microfluidic device may further comprise amplification of a nucleic acid sample. The microfluidic device may be filled with an amplification reagent comprising nucleic acid molecules, components necessary for an amplification reaction, an indicator molecule, and an amplification probe. The amplification may be performed by thermal cycling the plurality of chambers. Detection of nucleic acid amplification may be performed by imaging the chambers of the microfluidic device. The nucleic acid molecules may be quantified by counting the chambers in which the nucleic acid molecules are successfully amplified and applying Poisson statistics. In some embodiments, nucleic acid amplification and quantification may be performed in a single integrated unit.

A variety of nucleic acid amplification reactions may be used to amplify the nucleic acid molecule in a sample to generate an amplified product. Amplification of a nucleic acid target may be linear, exponential, or a combination thereof. Non-limiting examples of nucleic acid amplification methods include primer extension, polymerase chain reaction, reverse transcription, isothermal amplification, ligase chain reaction, helicase-dependent amplification, asymmetric amplification, rolling circle amplification, and multiple displacement amplification. In some embodiments, the amplification product is DNA or RNA. For embodiments directed towards DNA amplification, any DNA amplification method may be employed. DNA amplification methods include, but are not limited to, PCR, real-time PCR, assembly PCR, asymmetric PCR, digital PCR, dial-out PCR, helicase-dependent PCR, nested PCR, hot start PCR, inverse PCR, methylation-specific PCR, miniprimer PCR, multiplex PCR, overlap-extension PCR, thermal asymmetric interlaced PCR, touchdown PCR, and ligase chain reaction. In some embodiments, DNA amplification is linear, exponential, or any combination thereof. In some embodiments, DNA amplification is achieved with digital PCR (dPCR).

Reagents necessary for nucleic acid amplification may include polymerizing enzymes, reverse primers, forward primers, and amplification probes. Examples of polymerizing enzymes include, without limitation, nucleic acid polymerase, transcriptase, or ligase (i.e., enzymes which catalyze the formation of a bond). The polymerizing enzyme can be naturally occurring or synthesized. Examples of polymerases include a DNA polymerase, and RNA polymerase, a thermostable polymerase, a wild-type polymerase, a modified polymerase, E. coli DNA polymerase I, T7 DNA polymerase, bacteriophage T4 DNA polymerase Φ29 (phi29) DNA polymerase, Taq polymerase, Tth polymerase, Tli polymerase, Pfu polymerase Pwo polymerase, VENT polymerase, DEEPVENT polymerase, Ex-Taq polymerase, LA-Taw polymerase, Sso polymerase Poc polymerase, Pab polymerase, Mth polymerase ES4 polymerase, Tru polymerase, Tac polymerase, Tne polymerase, Tma polymerase, Tca polymerase, Tih polymerase, Tfi polymerase, Platinum Taq polymerases, Tbr polymerase, Tfl polymerase, Pfutubo polymerase, Pyrobest polymerase, KOD polymerase, Bst polymerase, Sac polymerase, Klenow fragment polymerase with 3′ to 5′ exonuclease activity, and variants, modified products and derivatives thereof. For a Hot Start polymerase, a denaturation step at a temperature from about 92° C. to 95° C. for a time period from about 2 minutes to 10 minutes may be required.

In some embodiments, the amplification probe is a sequence-specific oligonucleotide probe. The amplification probe may be optically active when hybridized with an amplification product. In some embodiments, the amplification probe is only detectable as nucleic acid amplification progresses. The intensity of the optical signal may be proportional to the amount of amplified product. A probe may be linked to any of the optically-active detectable moieties (e.g., dyes) described herein and may also include a quencher capable of blocking the optical activity of an associated dye. Non-limiting examples of probes that may be useful as detectable moieties include TaqMan probes, TaqMan Tamara probes, TaqMan MGB probes, Lion probes, locked nucleic acid probes, or molecular beacons. Non-limiting examples of quenchers that may be useful in blocking the optical activity of the probe include Black Hole Quenchers (BHQ), Iowa Black FQ and RQ quenchers, or Internal ZEN Quenchers. Alternatively or in addition to, the probe or quencher may be any probe that is useful in the context of the methods of the present disclosure.

In some embodiments, the amplification probe is a dual labeled fluorescent probe. The dual labeled probe may include a fluorescent reporter and a fluorescent quencher linked with a nucleic acid. The fluorescent reporter and fluorescent quencher may be positioned in close proximity to each other. The close proximity of the fluorescent reporter and fluorescent quencher may block the optical activity of the fluorescent reporter. The dual labeled probe may bind to the nucleic acid molecule to be amplified. During amplification, the fluorescent reporter and fluorescent quencher may be cleaved by the exonuclease activity of the polymerase. Cleaving the fluorescent reporter and quencher from the amplification probe may cause the fluorescent reporter to regain its optical activity and enable detection. The dual labeled fluorescent probe may include a 5′ fluorescent reporter with an excitation wavelength maximum of about 450 nanometers (nm), 500 nm, 525 nm, 550 nm, 575 nm, 600 nm, 625 nm, 650 nm, 675 nm, 700 nm, or higher and an emission wavelength maximum of about 500 nm, 525 nm, 550 nm, 575 nm, 600 nm, 625 nm, 650 nm, 675 nm, 700 nm, or higher. The dual labeled fluorescent probe may also include a 3′ fluorescent quencher. The fluorescent quencher may quench fluorescent emission wavelengths between about 380 nm and 550 nm, 390 nm and 625 nm, 470 nm and 560 nm, 480 nm and 580 nm, 550 nm and 650 nm, 550 nm and 750 nm, or 620 nm and 730 nm.

In some embodiments, the nucleic acid amplification is performed by thermal cycling the chambers of the microfluidic device. Thermal cycling may include controlling the temperature of the microfluidic device by applying heating or cooling to the microfluidic device. Heating or cooling methods may include resistive heating or cooling, radiative heating or cooling, conductive heating or cooling, convective heating or cooling, or any combination thereof. Thermal cycling may include cycles of incubating the chambers at a temperature sufficiently high to denature nucleic acid molecules for a duration followed by incubation of the chambers at an extension temperature for an extension duration. Denaturation temperatures may vary depending upon, for example, the particular nucleic acid sample, the reagents used, and the desired reaction conditions. In some embodiments, a denaturation temperature may be from about 80° C. to about 110° C. In some embodiments, a denaturation temperature may be from about 85° C. to about 105° C. In some embodiments, a denaturation temperature may be from about 90° C. to about 100° C. In some embodiments, a denaturation temperature may be from about 90° C. to about 98° C. In some embodiments, a denaturation temperature may be from about 92° C. to about 95° C. In some embodiments, a denaturation temperature may be at least about 80° C., at least about 81° C., at least about 82° C., at least about 83° C., at least about 84° C., at least about 85° C., at least about 86° C., at least about 87° C., at least about 88° C., at least about 89° C., at least about 90° C., at least about 91° C., at least about 92° C., at least about 93° C., at least about 94° C., at least about 95° C., at least about 96° C., at least about 97° C., at least about 98° C., at least about 99° C., at least about 100° C., or higher.

The duration for denaturation may vary depending upon, for example, the particular nucleic acid sample, the reagents used, and the desired reaction conditions. In some embodiments, the duration for denaturation (e.g., denaturation dwell time) may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. In an alternative embodiment, the duration for denaturation may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. In an example, the duration (e.g., dwell time) for denaturation may be less than or equal to about 2 seconds.

Extension temperatures (e.g., elongation temperatures) may vary depending upon, for example, the particular nucleic acid sample, the reagents used, and the desired reaction conditions. In some embodiments, an extension temperature may be from about 30° C. to about 80° C. In some embodiments, an extension temperature may be from about 35° C. to about 75° C. In some embodiments, an extension temperature may be from about 45° C. to about 65° C. In some embodiments, an extension temperature may be from about 55° C. to about 65° C. In some embodiments, an extension temperature may be from about 40° C. to about 60° C. In some embodiments, an extension temperature may be at least about 35° C., at least about 36° C., at least about 37° C., at least about 38° C., at least about 39° C., at least about 40° C., at least about 41° C., at least about 42° C., at least about 43° C., at least about 44° C., at least about 45° C., at least about 46° C., at least about 47° C., at least about 48° C., at least about 49° C., at least about 50° C., at least about 51° C., at least about 52° C., at least about 53° C., at least about 54° C., at least about 55° C., at least about 56° C., at least about 57° C., at least about 58° C., at least about 59° C., at least about 60° C., at least about 61° C., at least about 62° C., at least about 63° C., at least about 64° C., at least about 65° C., at least about 66° C., at least about 67° C., at least about 68° C., at least about 69° C., at least about 70° C., at least about 71° C., at least about 72° C., at least about 73° C., at least about 74° C., at least about 75° C., at least about 76° C., at least about 77° C., at least about 78° C., at least about 79° C., or at least about 80° C.

Extension time (e.g., elongation dwell time) may vary depending upon, for example, the particular nucleic acid sample, the reagents used, and the desired reaction conditions. In some embodiments, the duration for extension may be less than or equal to about 300 seconds, 240 seconds, 180 seconds, 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. In an alternative embodiment, the duration for extension may be no more than about 120 seconds, 90 seconds, 60 seconds, 55 seconds, 50 seconds, 45 seconds, 40 seconds, 35 seconds, 30 seconds, 25 seconds, 20 seconds, 15 seconds, 10 seconds, 5 seconds, 2 seconds, or 1 second. In an example, the duration for the extension reaction (e.g., elongation dwell time) is less than or equal to about 10 seconds.

Nucleic acid amplification may include multiple cycles of thermal cycling. Any suitable number of cycles may be performed. In some embodiments, the number of cycles performed may be more than about 5, more than about 10, more than about 15, more than about 20, more than about 30, more than about 40, more than about 50, more than about 60, more than about 70, more than about 80, more than about 90, more than about 100 cycles, or more. The number of cycles performed may depend upon the number of cycles necessary to obtain detectable amplification products. For example, the number of cycles necessary to detect nucleic acid amplification during dPCR may be less than or equal to about 100, less than or equal to about 90, less than or equal to about 80, less than or equal to about 70, less than or equal to about 60, less than or equal to about 50, less than or equal to about 40, less than or equal to about 30, less than or equal to about 20, less than or equal to about 15, less than or equal to about 10, less than or equal to about 5 cycles, or less. In an example, less than or equal to about 40 cycles are used and the total cycle time is less than or equal to about 20 minutes.

The time to reach a detectable amount of amplification product may vary depending upon the particular nucleic acid sample, the reagents used, the amplification reaction used, the number of amplification cycles used, and the desired reaction conditions. In some embodiments, the time to reach a detectable amount of amplification product may be about 120 minutes or less, 90 minutes or less, 60 minutes or less, 50 minutes or less, 40 minutes or less, 30 minutes or less, 20 minutes or less, 10 minutes or less, or 5 minutes or less. In an example, a detectable amount of amplification product may be reached in less than 20 minutes.

In some embodiments, the ramping rate (i.e., the rate at which the chamber transitions from one temperature to another) is important for amplification. For example, the temperature and time for which an amplification reaction yields a detectable amount of amplified product may vary depending upon the ramping rate. The ramping rate may impact the time(s), temperature(s), or both the time(s) and temperature(s) used during amplification. In some embodiments, the ramping rate is constant between cycles. In some embodiments, the ramping rate varies between cycles. The ramping rate may be adjusted based on the sample being processed. For example, optimum ramping rate(s) may be selected to provide a robust and efficient amplification method.

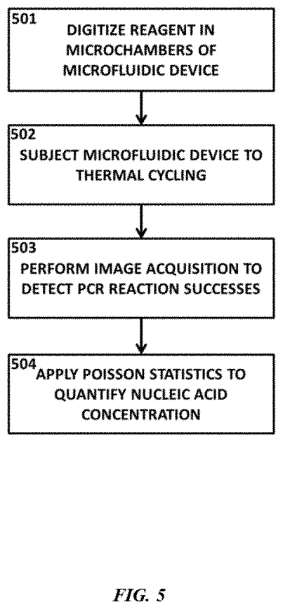

FIG. 5 illustrates a digital PCR process to be employed with the above-described microfluidic device. In step 501, reagent is partitioned as shown in FIGS. 3A-3D. In step 502, the reagent is subjected to thermal cycling to run the PCR reaction on the reagent in the chambers. This step may be performed, for example, using a flat block thermal cycler. In step 503, image acquisition is performed to determine which chambers have successfully run the PCR reaction. Image acquisition may, for example, be performed using a three color probe detection unit. In step 504, Poisson statistics are applied to the count of chambers determined in step 503 to convert the raw number of positive chambers into a nucleic acid concentration.

### System for Analyzing a Nucleic Acid Sample

In an aspect, the present disclosure provides a system for thermal cycling a microfluidic device. The system may comprise a microfluidic device, a thermal module, a pneumatic module (e.g., or pneumatic manifold), and one or more computer processors coupled to the thermal module and pneumatic module. The microfluidic device may comprise a plurality of chambers. A chamber of the plurality of chambers may comprise a nucleic acid sample comprising at least one nucleic acid molecule. The microfluidic device may have one or more surfaces that comprise a film (e.g., thin film), barrier, or membrane. The thermal module may be in thermal communication with the film or barrier. Alternatively, or in addition to, the thermal module may be in thermal communication with a surface opposite of the film or barrier (e.g., the other side of the microfluidic device from the film or barrier). The thermal module may be configure to thermal cycle or may thermal cycle the plurality of chambers at such a rate that a single round of thermal cycling (e.g., heating and cooling) is completed in 20 seconds or less. The thermal module may be configured to maintain or may maintain a temperature across the plurality of chambers that is within 0.2° C. The pneumatic module may be in fluid communication with the microfluidic device and may be configured to load the nucleic acid samples into the chambers of the microfluidic device. The pneumatic module may additionally be configured to apply pressure to the microfluidic device to maintain thermal contact between the film or barrier of the microfluidic device and the thermal module. Alternatively, or in addition to, the film or barrier may contact the thermal module to provide thermal contact. The one or more computer processor may be configured or otherwise programmed to direct the pneumatic module to load the nucleic acid sample into the chambers, direct the pneumatic module to apply pressure to the microfluidic device to maintain thermal contact, or physical contact, between the film or barrier of the microfluidic device and the thermal module, and direct the thermal module to thermal cycle the plurality of chambers to amplify the nucleic acid molecule(s) in the chambers.

In another aspect, the present disclosure provides a system for thermal cycling a microfluidic device. The system may comprise a microfluidic device, a thermal module, a pneumatic module (e.g., or pneumatic manifold), and one or more computer processors coupled to the thermal module and pneumatic module. The microfluidic device may comprise a plurality of chambers. A chamber of the plurality of chambers may comprise a nucleic acid sample comprising at least one nucleic acid molecule. The microfluidic device may have one or more surfaces that comprise a film (e.g., thin film), barrier, or membrane. The film, barrier, or membrane may have a thermal conductivity that is less than or equal to about 1 watt per meter Kelvin (W/m-K). The thermal module may be in thermal communication with the film or barrier. Alternatively, or in addition to, the thermal module may be in thermal communication with a surface opposite of the film or barrier (e.g., the other side of the microfluidic device from the film or barrier). The thermal module may be configure to thermal cycle or may thermal cycle the plurality of chambers at such a rate that a single round of thermal cycling (e.g., heating and cooling) is completed in 20 seconds or less. The pneumatic module may be in fluid communication with the microfluidic device and may be configured to load the nucleic acid samples into the chambers of the microfluidic device. The pneumatic module may additionally be configured to apply pressure to the microfluidic device to maintain thermal contact between the film or barrier of the microfluidic device and the thermal module. Alternatively, or in addition to, the film or barrier may contact the thermal module to provide thermal contact. The one or more computer processor may be configured or otherwise programmed to direct the pneumatic module to load the nucleic acid sample into the chambers, direct the pneumatic module to apply pressure to the microfluidic device to maintain thermal contact, or physical contact, between the film or barrier of the microfluidic device and the thermal module, and direct the thermal module to thermal cycle the plurality of chambers to amplify the nucleic acid molecule(s) in the chambers.

The system may be configured such that a single round of thermal cycling (e.g., heating and cooling, or extension and denaturation) may be performed in less than or equal to about 90 seconds, 80 seconds, 70 second, 60 second, 50, seconds, 40 seconds, 30 seconds, 20 seconds, 10 seconds, 5 seconds, or less. In an example a single round of thermal cycling may be completed in less than or equal to about 30 seconds. In another example, a single round of thermal cycling may be completed in less than or equal to about 20 seconds. In another example, a single round of thermal cycling may be completed in less than or equal to about 10 seconds. Thermal cycling the chambers may permit activation of a primer extension reaction or nucleic acid amplification reaction (e.g., polymerase chain reaction).

In another aspect, the present disclosure provides an apparatus for using a microfluidic device to analyze nucleic acid samples. The apparatus may comprise a transfer stage configured to hold one or more microfluidic devices. The microfluidic devices may comprise a channel with an inlet and an outlet, a plurality of chambers connected to the channel by a plurality of siphon apertures, and a thin film capping or covering the microfluidic device. The apparatus may comprise a pneumatic module in fluid communication with the microfluidic device. The pneumatic module may load reagent into the microfluidic device and partition the reagent into the chambers. The apparatus may comprise a thermal module in thermal communication with the plurality of chambers. The thermal module may control the temperature of the chambers and thermal cycle the chambers. The apparatus may comprise an optical module capable of imaging the plurality of chambers. The apparatus may also comprise a computer processor coupled to the transfer stage, pneumatic module, thermal module, and optical module. The computer processor may be programmed to (i) direct the pneumatic module to load reagent into the microfluidic device and partition the reagent into the plurality of chambers, (ii) direct the thermal module to thermal cycle the plurality of chambers, and (iii) direct the optical module to image the plurality of chambers.

The transfer stage may be configured input the microfluidic device, hold the microfluidic device, and output the microfluidic device. The transfer stage may be stationary in one or more coordinates. Alternatively, or in addition to, the transfer stage may be capable of moving in the X-direction, Y-direction, Z-direction, or any combination thereof. The transfer stage may be capable of holding a single microfluidic device. Alternatively, or in addition to, the transfer stage may be capable of holding at least 2, at least 3, at least 4, at least 5, at least 6, at least 7, at least 8, at least 9, at least 10, or more microfluidic devices.

The pneumatic module may be a pneumatic manifold. The pneumatic module or manifold may be configured to be in fluid communication with the inlets and the outlets of the microfluidic device. The pneumatic module may have multiple connection points capable of connecting to multiple inlets and multiple outlets. The pneumatic module may be able to fill, backfill, and partition a single array of chambers at a time or multiple arrays of chambers in tandem. The pneumatic module may further comprise a vacuum module. The pneumatic module may provide increased pressure to the microfluidic device or provide vacuum to the microfluidic device.

The thermal module may be configured to be in thermal communication with the chambers of the microfluidic devices. The thermal module may be configured to control the temperature of a single array of chambers or to control the temperature of multiple arrays of chambers. The thermal control module may perform the same thermal program across all arrays of chambers or may perform different thermal programs with different arrays of chambers.

The system may further include a detection module. The detection module may provide electronic or optical detection. In an example, the detection module is an optical module providing optical detection (e.g., imaging of the microfluidic device). The optical module may be in optical communication and may image the plurality of chambers, the channel, the siphon apertures, or any combination thereof. The optical module may be configured to emit and detect multiple wavelengths of light. Emission wavelengths may correspond to the excitation wavelengths of the indicator and amplification probes used. The emitted light may include wavelengths with a maximum intensity around about 450 nm, 500 nm, 525 nm, 550 nm, 575 nm, 600 nm, 625 nm, 650 nm, 675 nm, 700 nm, or any combination thereof. Detected light may include wavelengths with a maximum intensity around about 500 nm, 525 nm, 550 nm, 575 nm, 600 nm, 625 nm, 650 nm, 675 nm, 700 nm, or any combination thereof. The optical module may be configured to emit one, two, three, four, or more wavelengths of light. The optical module may be configured to detect one, two, three, four, or more wavelengths of light. On emitted wavelength of light may correspond to the excitation wavelength of indicator molecule. Another emitted wavelength of light may correspond to the excitation wavelength of the amplification probe. One detected wavelength of light may correspond to the emission wavelength of an indicator molecule. Another detected wavelength of light may correspond to an amplification probe used to detect a reaction within the chambers. The optical module may be configured to image sections of an array of chambers. Alternatively, or in addition to, the optical module may image an entire array of chambers in a single image.

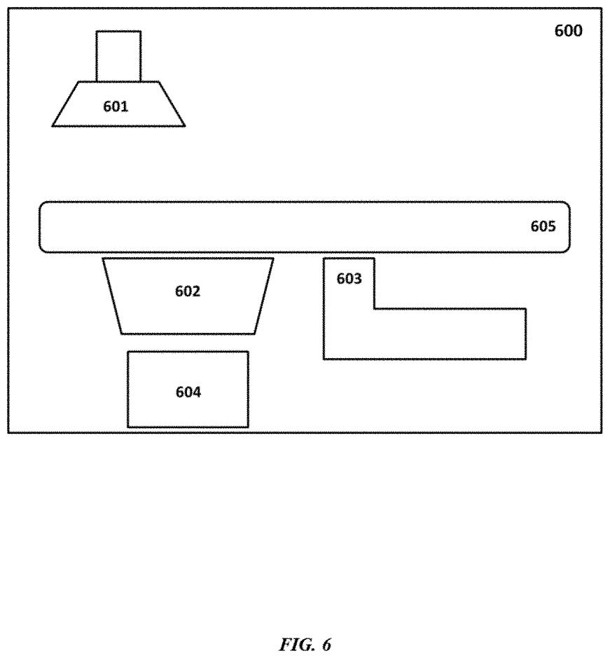

FIG. 6 illustrates a machine 600 for performing the process of FIG. 5 in a single machine. The machine 600 includes a pneumatic module 601, which contains pumps and manifolds and may be moved in a Z-direction, operable to perform the application of pressure as described in FIGS. 3A-3D. Machine 600 also includes a thermal module 602, such as a flat block thermal cycler, to thermally cycle the microfluidic device and thereby cause the polymerase chain reaction to run. Machine 600 further includes an optical module 603, such as an epi-fluorescent optical module, which can optically determine which chambers in the microfluidic device have successfully run the PCR reaction. The optical module 603 may feed this information to a processor 604, which uses Poisson statistics to convert the raw count of successful chambers into a nucleic acid concentration. A transfer stage 605 may be used to move a given microfluidic device between the various modules and to handle multiple microfluidic devices simultaneously. The microfluidic device described above, combined with the incorporation of this functionality into a single machine, reduces the cost, workflow complexity, and space requirements for dPCR over other implementations of dPCR.

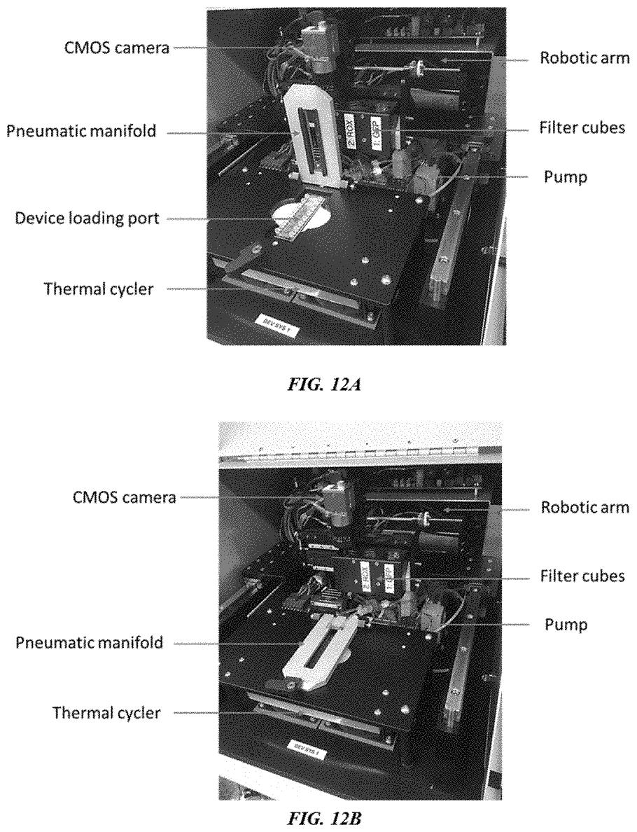

FIGS. 12A and 12B show an example system that may be used for dPCR, including dPCR with rapid thermal cycling. The system may include a stage, robotic arm, detection module, fluid flow module, and thermal module. The stage may be a platform or holder for the microfluidic device. The microfluidic device may be any microfluidic device described elsewhere herein. The robotic arm may move, alter, or arrange a position of the microfluidic device. Alternatively, or in addition to, the robotic arm may arrange or move other components of the system (e.g., fluid flow module or detection module). The detection module may include a camera (e.g., a complementary metal oxide semiconductor (CMOS) camera or a charge-coupled device (CCD) camera) and filter cubes. The filter cubes may alter or modify the wavelength of excitation light and/or the wavelength of light detected by the camera. The fluid flow module may comprise a manifold (e.g., pneumatic manifold) and/or one or more pumps. The manifold may be in an upright position, see FIG. 12A, such that the manifold does not contact the microfluidic device. The upright position may be used when loading and/or imaging the microfluidic device. The manifold may be in a downward position, see FIG. 12B, such that the manifold contacts the microfluidic device. The manifold may be used to load fluids (e.g., samples and reagents) into the microfluidic device. The manifold may apply a pressure to the microfluidic device to hold the device in place and/or to prevent warping, bending, or other stresses during use. In an example, the manifold applies a downward pressure and holds the microfluidic device against the thermal module.

The system may further comprise a thermal module. The thermal module may be configured to be in thermal communication with the chambers of the microfluidic device. The thermal module may be configured to control the temperature of a single array of chambers or to control the temperature of multiple arrays of chambers. Each array of chambers may be individually addressable by the thermal module. For example, the thermal module may perform the same thermal program across all arrays of chambers or may perform different thermal programs with different arrays of chambers. The thermal module may be in thermal communication with the microfluidic device and/or the chambers of the microfluidic device. The thermal module may heat or cool the microfluidic device. One or more surfaces of the microfluidic device may be in direct contact with the thermal module. Alternately, or in addition to, a thermally conductive material may be disposed between the thermal module and the microfluidic device. The thermal module may maintain the temperature across a surface of the microfluidic device such that the variation is less than or equal to about 2° C., 1.5° C., 1° C., 0.9° C., 0.8° C., 0.7° C., 0.6° C., 0.5° C., 0.4° C., 0.3° C., 0.2° C., 0.1° C., or less. In an example, the thermal module maintains the temperature across a surface of the microfluidic device within about 0.2° C. In another example, the thermal module maintains the temperature across the surface of the microfluidic device within about 0.1° C. The thermal module may maintain a temperature of a surface of the microfluidic device that is within about plus or minus 0.5° C., 0.4° C., 0.3° C., 0.2° C., 0.1° C., 0.05° C., or closer to a temperature set point.

The system may further include one or more computer processors. The one or more computer processors may be operatively coupled to the fluid flow module, holder, thermal module, detection module, robotic arm, or any combination thereof. In an example, the one or more computer processors is operatively coupled to the fluid flow module. The one or more computer processors may be individually or collectively programmed to direct the fluid flow module to supply a pressure differential to the inlet port when the fluid flow module is fluidically coupled to the inlet port to subject the solution to flow from the inlet port to the channel and/or from the channel to the chambers and, thereby, partition through pressurized out-gassing of the chambers. The one or more computer processors may be operatively coupled to the detection module and may programmed or otherwise configured to image the chambers at one or more time points during sample processing. For example, the one or more computer processors may direct the detection module to image the chambers during sample loading, after sample loading, during thermal cycling, and after thermal cycling. The one or more computer processors may additionally be programmed or otherwise configured for data processing. For example, the one or more computer processors may be programmed or otherwise configured to store images of the chambers, compare time point images of the chamber, generate data related to the images (e.g., fluorescence intensity, change in fluorescence intensity, etc.), and output the data to a user via a user interface and/or a display.

The present disclosure is not to be limited in scope by the specific embodiments described herein. Indeed, other various embodiments of and modifications to the present disclosure, in addition to those described herein, will be apparent to those of ordinary skill in the art from the foregoing description and accompanying drawings.

For example, while described in the context of a dPCR application, other microfluidic devices which may require a number of isolated chambers filled with a liquid, that are isolated via a gas or other fluid, may benefit from the use of a thin thermoplastic film to allow outgassing to avoid gas fouling while also providing an advantage with respect to manufacturability and cost. Other than PCR, other nucleic acid amplification methods such as loop mediated isothermal amplification can be adapted to perform digital detection of specific nucleic acid sequences according to embodiments of the present disclosure. The chambers can also be used to isolate single cells with the siphoning apertures designed to be close to the diameter of the cells to be isolated. In some embodiments, when the siphoning apertures are much smaller than the size of blood cells, embodiments of the present disclosure can be used to separate blood plasma from whole blood.

### Computer Systems for Analyzing a Nucleic Acid Sample

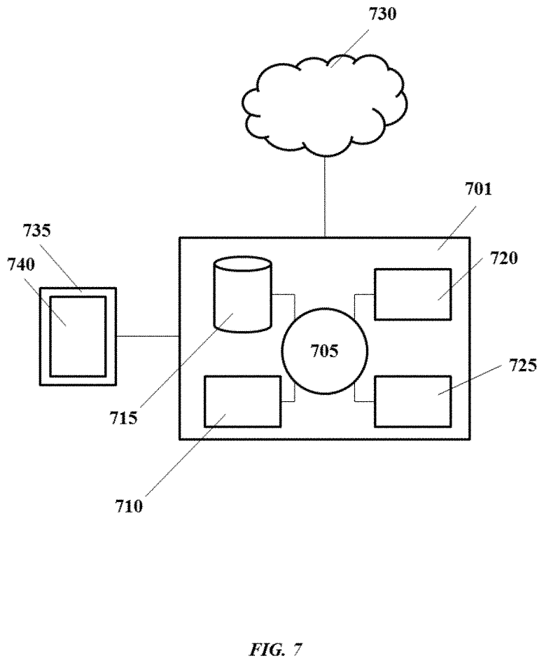

The present disclosure provides computer control systems that are programmed to implement methods of the disclosure. FIG. 7 shows a computer system 701 that can be programmed or otherwise configured for nucleic acid sample processing and analysis, including sample partitioning, amplification, and detection. The computer system 701 can regulate various aspects of methods and systems of the present disclosure. The computer system 701 can be an electronic device of a user or a computer system that can be remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage and/or electronic display adapters. The memory 710, storage unit 715, interface 720 and peripheral devices 725 are in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that can be in communication with the Internet. The network 730 in some cases can be a telecommunication and/or data network. The network 730 can include one or more computer servers, which can enable distributed computing, such as cloud computing. The network 730, in some cases with the aid of the computer system 701, can implement a peer-to-peer network, which may enable devices coupled to the computer system 701 to behave as a client or a server.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods of the present disclosure. Examples of operations performed by the CPU 705 can include fetch, decode, execute, and writeback.

The CPU 705 can be part of a circuit, such as an integrated circuit. One or more other components of the system 701 can be included in the circuit. In some cases, the circuit is an application specific integrated circuit (ASIC).

The storage unit 715 can store files, such as drivers, libraries and saved programs. The storage unit 715 can store user data, e.g., user preferences and user programs. The computer system 701 in some cases can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some cases, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code, or can be compiled during runtime. The code can be supplied in a programming language that can be selected to enable the code to execute in a pre-compiled or as-compiled fashion.

In one aspect, the present disclosure provides a non-transitory computer-readable medium comprising machine executable code that, upon execution by one or more computer processors, implements a method for forming a microfluidic device to amplify and quantify a nucleic acid sample. The method may comprise: injection molding thermoplastic to create a microfluidic structure comprising at least one channel, a plurality of chambers, and a plurality of siphon apertures, wherein the plurality of chambers are connected to the at least one channel by the plurality of siphon apertures; forming at least one inlet and at least one outlet, wherein the at least one inlet and the at least one outlet are in fluid communication with the at least on channel; and applying a thermoplastic thin film to cap the microfluidic structure, wherein the thermoplastic thin film is at least partially gas permeable to a pressure differential is applied across the thermoplastic thin film.

In one aspect, the present disclosure provides a non-transitory computer-readable medium comprising machine executable code that, upon execution by one or more computer processors, implements a method for analyzing and quantifying a nucleic acid sample. The method may comprise: providing the microfluidic device comprising at least one channel, wherein the at least one channel comprises at least one inlet and at least one outlet, and wherein the microfluidic device further comprises a plurality of chambers connected to the channel by a plurality of siphon apertures, and a thermoplastic thin film disposed adjacent to a surface of the microfluidic device such that the thermoplastic thin film caps the channel, the plurality of chambers, and the plurality of siphon apertures; providing a reagent to the at least one inlet or to the at least one outlet; filling the microfluidic device by providing a first pressure differential between the reagent and the microfluidic device, wherein the first pressure differential causes the reagent to flow into the microfluidic device; applying a second pressure differential between the channel and the plurality of chambers to move the reagent into the plurality of chambers and to force gas within the plurality of chambers to pass through the thermoplastic thin film capping or covering the plurality of chambers, the plurality of siphon apertures, and the channel, wherein the second pressure differential is greater than the first pressure differential; and applying a third pressure differential between the at least one inlet and the at least one outlet to introduce a fluid into the channel without introducing the fluid into the chambers, wherein the third pressure differential is less than the second pressure differential.

Aspects of the systems and methods provided herein, such as the computer system 701, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 701 can include or be in communication with an electronic display 735 that comprises a user interface (UI) 740 for providing, for example, depth profile of an epithelial tissue. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 705. The algorithm can, for example, regulate systems or implement methods provided herein.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

### Example 1: Demonstration of Reagent Partitioning

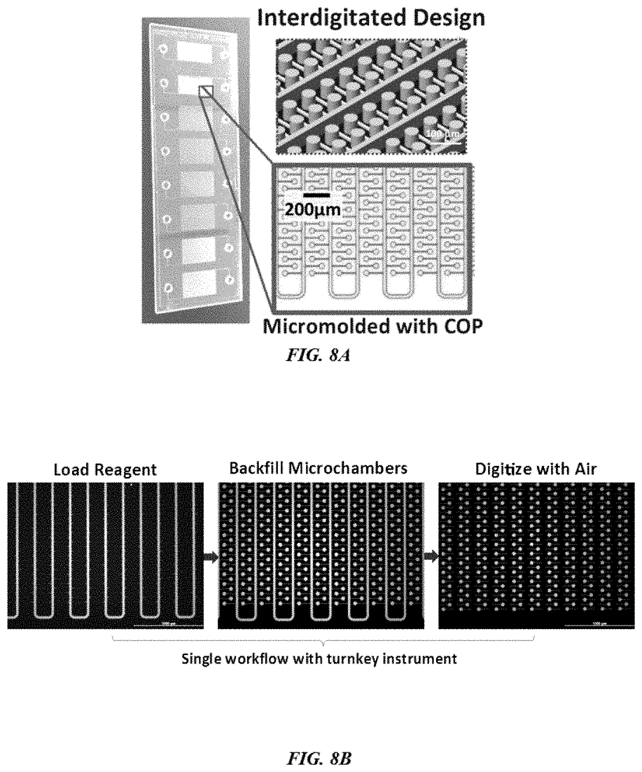

Reagent partitioning is demonstrated using a microfluidic device fabricated using standard microscope slide dimensions. The total dimensions of the microfluidic device are 1 inch wide, 3 inches long, and 0.6 inches thick. The device contains four different chamber array designs and a total of eight different arrays of chambers. FIG. 8A shows the eight-unit device and an enlarged perspective of one of the four array designs. The microfluidic device is molded from a cyclo-olefin polymer (COP), Zeonor 790R (Zeon Chemicals, Japan) and sealed by thermal bonding with a 100 μm COP thin film, Zeonox ZF14 (Zeon Chemicals, Japan). The shown enlarged microfluidic segment has a serpentine channel connected to chambers by siphon apertures. The chambers are in a gridded configuration. The chambers and channel have a depth of 40 μm the siphon apertures have a depth of 10 μm. Each isolated microfluidic segment has an inlet and an outlet channel. The inlet and outlet channels are mechanically drilled before the film is thermally bonded to the base of the microfluidic device. The inlet and outlet channels are 1.6 mm in diameter.

FIG. 8B shows fluorescent images of reagent loading, chamber backfilling, and partitioning. Prior to loading the microfluidic device 2 microliters (μL) of a 4 kiloDalton (kDa) fluorescein conjugated dextran (Sigma-Aldrich, St. Louis, Mo.) is pipetted into the inlet. The microfluidic device is then contacted with a pneumatic controller. The pneumatic controller loads the channel of the microfluidic device by applying 4 psi of pressure to the inlet for 3 minutes. The chambers are filled by pressurizing both the inlet and the outlet to 10 psi for 20 minutes. The reagent is then partitioned by flowing air at 4 psi from the inlet of the microfluidic device to clear reagent from the channel.

### Example 2: Single Instrument Workflow for dPCR

The methods for amplification and quantification of nucleic acids in the microfluidic device may be performed in a single instrument. The instrument may be capable of reagent partitioning, thermal cycling, image acquisition, and data analysis. FIG. 9 shows a prototype instrument capable of a single instrument work flow. The instrument is designed to accommodate up to four devices at a time and enable concurrent image acquisition and thermal cycling. The instrument contains a pneumatic module for reagent partitioning, a thermal module for temperature control and thermal cycling, an optical module for imaging, and a scanning module. The optical module has two fluorescent imaging capabilities and is able to detect fluorescent emissions of approximately 520 nm and 600 nm, which correspond to the emission wavelengths of FAM and ROX fluorophores, respectively. The optical module has a 25 mm by 25 mm field of view and a Numerical Aperture (NA) of 0.14.

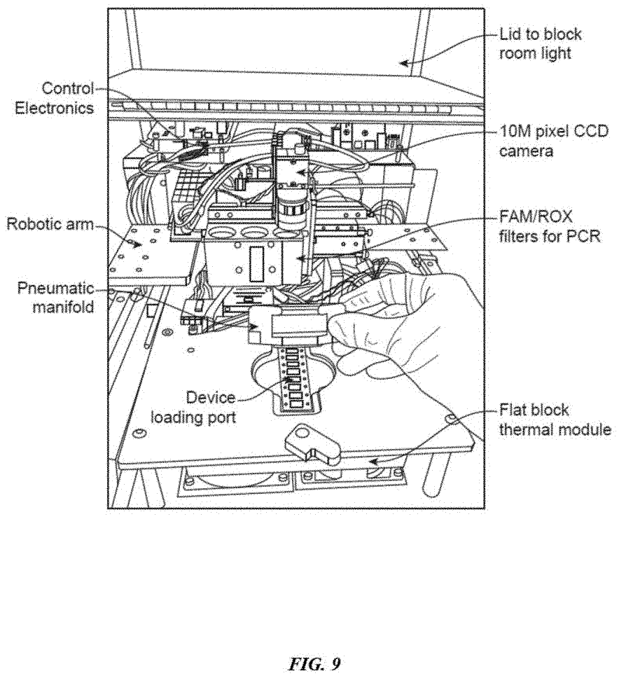

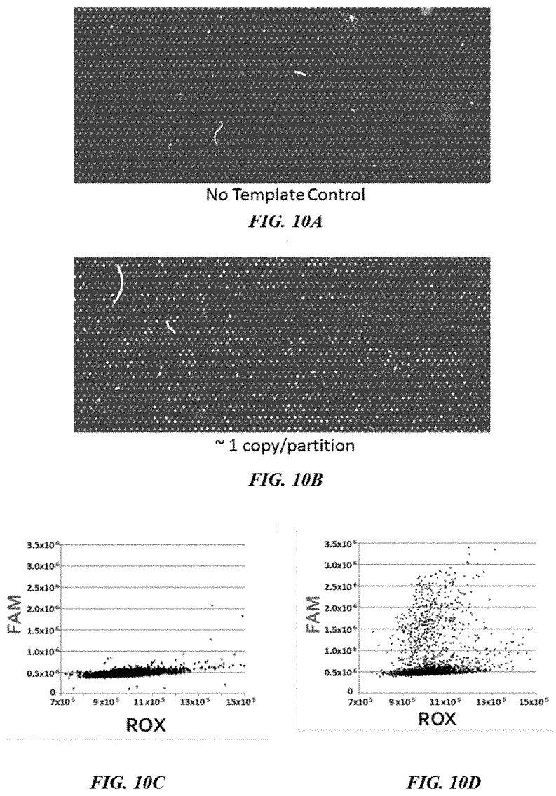

The single instrument workflow may be tested using a well-established qPCR assay utilizing a TaqMan probe as a reporter. Briefly, a nucleic acid sample is mixed with PCR reagents. The PCR reagents include forward primers, reverse primers, TaqMan probes, and a ROX indicator. The sequence of the forward primer is 5′-GCC TCA ATA AAG CTT GCC TTG A-3′. The sequence of the reverse primer is 5′-GGG GCG CAC TGC TAG AGA-3′. The sequence of the TaqMan probe is 5′-[FAM]-CCA GAG TCA CAC AAC AGA CGG GCA CA-[BHQ1]-3′. The nucleic acid sample and PCR reagents are loaded and partitioned within the microfluidic device following the above mentioned protocol. PCR amplification is performed by increasing the temperature of the chambers to 95° C. and holding the temperature for 10 minutes followed by forty cycles ramping the temperature of the chambers from 95° C. to 59° C. at a rate of 2.4° C. per second with a 1 minute hold at 59° C. prior to returning the temperature to 95° C. FIGS. 10A-10D show fluorescent images of samples containing approximately one nucleic acid template copy per partition and partitions containing zero nucleic acid template copies per partition (no template control or NTC) after PCR amplification and fluorescence intensity plots of samples containing approximately one nucleic acid copy per partition and NTC partitions after PCR amplification. FIG. 10A shows a fluorescent image of the partitioned sample containing no nucleic acid template and each grey dot represents a single chamber containing the PCR reagents. The image is taken by exciting the ROX indicator within each chamber with approximately 575 nm light and imaging the emission spectrum, which has a max emission at approximately 600 nm. FIG. 10B shows the partitioned sample containing approximately one nucleic acid template copy per partition after PCR amplification. After PCR amplification, imaging shows chambers that contain the ROX indicator and chambers that contain both the ROX indicator and emission from the FAM probe. The FAM probe has an excitation wavelength of approximately 495 nm and an emission wavelength maximum of approximately 520 nm. Individual chambers contain the ROX indicator, the FAM probe, and the BHQ-1 quencher. As with FIG. 10A each grey dot represents a chamber containing the partitioned sample with no nucleic acid template. The white dots represent chambers that contain nucleic acid samples that have been successfully amplified. Upon successful PCR amplification, the FAM fluorophore and BHQ-1 quencher may be cleaved from the TaqMan probe, resulting in a detectable fluorescent signal. FIGS. 10C and 10D show a 2-dimensional scatter plot of the FAM fluorescent intensity as a function of the ROX fluorescent intensity for each chamber for the partitioned and amplified microfluidic device, respectively. FIG. 10C shows a sample containing zero nucleic acid templates per partition, resulting in a FAM fluorescent intensity that is predominantly constant over a range of ROX fluorescent intensities. FIG. 10D shows a sample containing approximately one nucleic acid template copy per partition, resulting in a FAM fluorescent intensity that varies as a function of ROX fluorescent intensity due to the presence of amplification signals within the partition.

### Example 3: Polymerase Chain Reaction Using Rapid Thermal Cycling

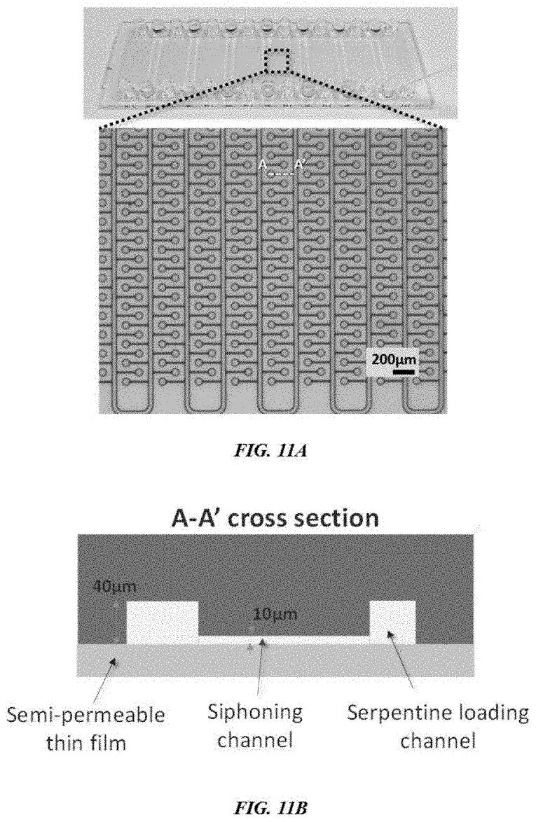

The microfluidic devices and systems described elsewhere herein may be used for rapid thermal cycling. Rapid thermal cycling may be used to activate and control a dPCR reaction. FIGS. 11A and 11B show an example microfluidic device for rapid thermal cycling. FIG. 11A shows an image of the microfluidic device and with a section of the microfluidic device expanded. The microfluidic device may be formed to be approximately the size of a microscope slide. The microfluidic device may have one or more sections. The sections may not be in fluid communication with one another. Each section may have a fluid inlet and a fluid outlet. Each section may have a microfluidic channel in fluid communication with a plurality of chambers. FIG. 11B illustrates a cross sectional view of an example chamber from FIG. 11A. The chambers may have a height of approximately 40 μm. The channel may have the same height as the chambers. The siphon aperture may have a height of approximately 10 μm. The chambers, siphon aperture, and fluid flow channel may be covered by a semi-permeable thin film. The semi-permeable thin film may allow off gassing when there is a large pressure differential between the surfaces of the thin film. The microfluidic device may be loaded with the reagents for dPCR. The microfluidic device may be cycled to activate and control the dPCR reaction.

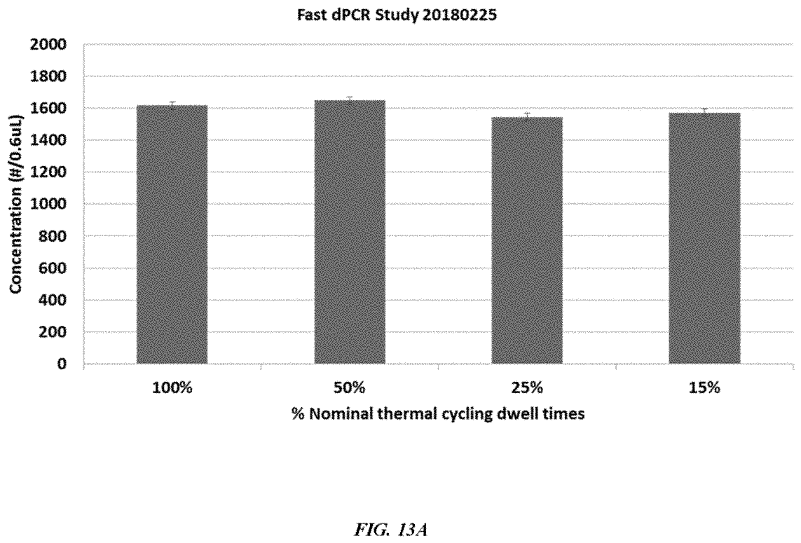

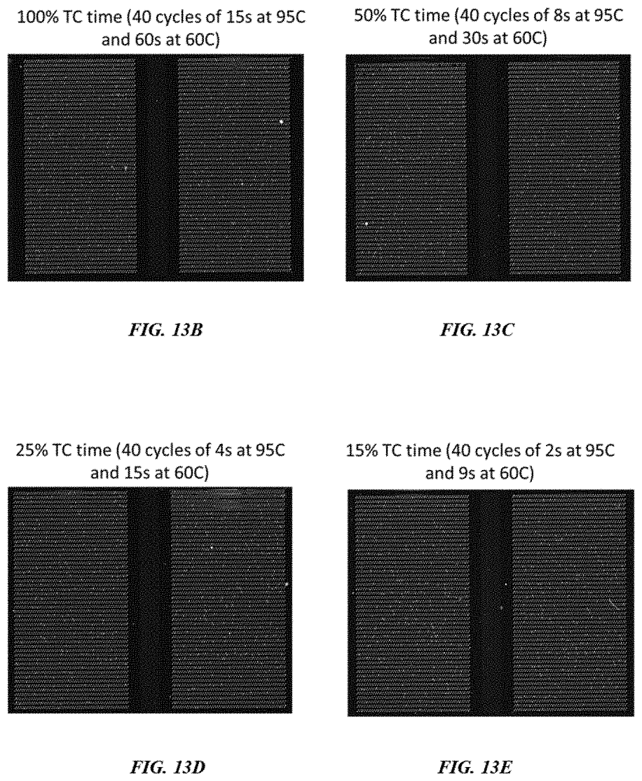

FIGS. 13A-13E shows dPCR results as a function of thermal cycling duration. Four different dPCR reactions are performed. Each reaction is thermal cycled forty times over the course of the experiment. Each reaction is thermal cycled using a different dwell time. The dwell time may be the time at which a reaction sits at a given temperature. For example, in this experiment, the two temperatures used are about 95° C. for denaturation and about 60° C. for elongation. During each cycle, the reaction is maintained at the higher temperature for one dwell time and the lower temperature for a second dwell time. During each cycle, the temperatures and dwell time are maintained constant. FIG. 13A shows a plot of nucleic acid template concentration as a function of percent change in dwell time. Decreasing the dwell times by eighty five percent is seen to not affect the concentration of nucleic acid template after forty cycles as compared to a sample without a reduced dwell time. FIGS. 13B-13E show the fluorescence images of each of the reactions after completion of forty thermal cycles. FIG. 13B shows fluorescence results after completing forty cycles of maintaining the sample at 95° C. for fifteen seconds followed by sixty seconds at 60° C. These dwell times represent a one hundred percent, or standard, thermal cycle program. FIG. 13C shows fluorescence results after completing forty cycles of maintaining the sample at 95° C. for eight seconds followed by thirty seconds at 60° C., representing a fifty percent decrease in dwell time. FIG. 13D shows fluorescence results after completing forty cycles of maintaining the sample at 95° C. for four seconds followed by fifteen seconds at 60° C., representing a seventy-five percent decrease in dwell time. FIG. 13E shows fluorescence results after completing forty cycles of maintaining the sample at 95° C. for two seconds followed by nine seconds at 60° C., representing a eighty-five percent decrease in dwell time.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-52. (canceled)

53. A method for thermal cycling a microfluidic device, comprising:
(a) providing a microfluidic device that is in fluid communication with a pneumatic module and that is in thermal communication with a thermal module, wherein said microfluidic device comprises a plurality of chambers, wherein said microfluidic device comprises a film or barrier that seals said plurality of chambers, wherein said film or barrier has a thermal conductivity of less than or equal to about 1 watt per meter Kelvin (W/m-K) at 20° C., and wherein said film or barrier is in thermal communication with said thermal module;
(b) using said pneumatic module to load a nucleic acid sample comprising at least one nucleic acid molecule into a chamber of said plurality of chambers of said microfluidic device;
(c) using said pneumatic module to apply a pressure to said microfluidic device to maintain thermal contact between said film or barrier of said microfluidic device and said thermal module; and
(d) using said thermal module to thermal cycle said plurality of chambers to amplify said at least one nucleic acid molecule in said chamber, wherein a single round of thermal cycling is completed within about 20 seconds or less.

54. The method of claim 53, wherein thermal cycling said plurality of chambers activates a polymerase chain reaction.

55. The method of claim 53, wherein at least forty cycles of said polymerase chain reaction are completed in less than twenty minutes.

56. The method of claim 53, wherein said film or barrier has a thermal conductivity of less than or equal to about 0.5 W/m-K at 20° C.

57. The method of claim 56, wherein said film or barrier has a thermal conductivity of less than or equal to about 0.2 W/m-K at 20° C.

58. The method of claim 53, wherein said film or barrier comprises a polymeric material.

59. The method of claim 53, further comprising using an optical module in optical communication with said plurality of chambers to image said plurality of chambers.

60. The method of claim 53, wherein said film or barrier has a thickness of less than or equal to about 250 micrometers (μm).

61. (canceled)

62. The method of claim 53, further comprising using said pneumatic module to apply a pressure differential across said film or barrier.

63. The method of claim 53, wherein a chamber of said plurality of chambers has a depth of less than or equal to about 50 μm.

64. The method of claim 53, wherein said film or barrier contacts a surface of said thermal module.

65. The method of claim 53, wherein using said pneumatic module to apply pressure to said microfluidic device prevents warping of said microfluidic device during thermal cycling.

66. The method of claim 53, wherein said microfluidic device further comprises at least one channel in fluid communication with said plurality of chambers.

67. The method of claim 66, wherein said microfluidic device further comprises a plurality of siphon apertures, and wherein said plurality of siphon apertures provide said fluid communication between said at least one channel and said plurality of chambers.

68. The method of claim 67, further comprising using said pneumatic module to apply a first pressure to said at least one channel to load a sample into said at least one channel.

69. The method of claim 68, further comprising using said pneumatic module to apply a second pressure to said at least one channel to load said sample into said plurality of chambers.

70.-78. (canceled)

79. The method of claim 53, wherein said chamber has a volume of less than or equal to about 150 picoliters (pL).
